(19) United States
(12) Patent Application Publication
(10) Pub. No.: US 2015/0289961 A1
(43) Pub. Date: Oct. 15, 2015

(54) Adhesive Sheet for Teeth

(71) Applicant: KAO CORPORATION, Chuo-ku, Tokyo (JP)

(72) Inventors: Tomokazu TOGO, Sumida-ku, Tokyo (JP); Gen NAKAUCHI, Sumida-ku, Tokyo (JP); Ryota SHIIBA, Sumida-ku, Tokyo (JP)

(73) Assignee: Kao Corporation, Chuo-ku, Tokyo (JP)

(21) Appl. No.: 14/439,426

(22) PCT Filed: Oct. 31, 2013
(86) PCT No.: PCT/JP2013/079614

§ 371 Date: Apr. 29, 2015

(30) Foreign Application Priority Data
Oct. 31, 2012 (JP) 2012-241361

(51) Int. Cl. A61C 19/06; A61K 8/55; A61Q 11/00; A61K 8/73; A61K 8/46; A61K 8/02; A61K 8/24

(52) U.S. Cl. CPC A61C 19/066; A61K 8/0204; A61K 8/55; A61K 8/24; A61K 8/731; A61K 8/463; A61Q 11/00; A61K 2800/87; A61K 2800/92

(57) ABSTRACT

The present invention relates to an adhesive sheet for teeth which has high safety and makes it possible to obtain excellent tooth whitening effect and tooth gloss effect by a simple means of just being attached to teeth. The adhesive sheet for teeth comprises: a layer (A) that has a pH of 6.6 or more and 10.5 or less when the layer (A) is dissolved in water, and a layer (B) that comprises a component (b) of one or more kinds selected from phytic acid, hexamethaphosphoric acid, an organic acid having a pKa of 4.5 to 7.0 at 25° C. and salts thereof, and that has a pH of 3.5 or more and 6.5 or less when the layer (B) is dissolved in water.

### FIELD OF THE INVENTION

The present invention relates to an adhesive sheet for teeth that is excellent in a tooth whitening effect.

### BACKGROUND OF THE INVENTION

Human teeth are stained by adhesion of various staining substances on their surfaces as well as calculus and plaque, and the gloss of teeth also decreases. These staining and a decrease in gloss are not cosmetically desirable, and various means to whiten teeth are developed.

For example, Patent Document 1 discloses a tooth whitening patch having a multi-layer structure of a contact adhesive layer, an active substance-storage layer and a support layer, wherein the contact adhesive layer exerts adhesive force when the tooth whitening patch is attached to teeth, and the peroxide contained in the active substance-storage layer causes the tooth whitening action. In addition, Patent Document 2 discloses a multi-layer strip including a first film which has a first whitening agent and a first water-soluble or water-dispersible polymers, and a second film which has a second whitening agent and a second water-soluble or water-dispersible polymers, and describes different modes from each other of whitening agents such as hydrogen peroxide contained in the respective films.

On the other hand, Patent Document 3 discloses a method of producing a laminate including individual layers, and also describes that at least one layer in such laminate may contain at least one active substance and the like for a medicine or cosmetics, and a coating composition used in forming the layers may have different concentration, viscosity, cross-linkage, solid content, solvent content or pH.

### CITATION LIST

### Patent Document

Patent Document 1: JP 2004-534809 A

Patent Document 2: JP 2007-531771 A

Patent Document 3: JP 2003-524589 A

### SUMMARY OF THE INVENTION

The present invention provides an adhesive sheet for teeth comprising

a layer (A) which has a pH of 6.6 or more and 10.5 or less when the layer is dissolved in water, and

a layer (B) which comprises one or more ingredients (b) selected from phytic acid, hexamethaphosphoric acid, an organic acid which has a pKa of from 4.5 to 7.0 at 25° C. and salts thereof, and which has a pH of 3.5 or more and 6.5 or less when the layer is dissolved in water (hereinafter, also referred to as “the adhesive sheet of the present invention”).

### BRIEF DESCRIPTION OF DRAWINGS

FIG. 1 is a graph representing time-dependent pH changes in the laminated sheets of Examples 46 to 49 after the storage based on the measurement method 1.

FIG. 2 is a graph representing time-dependent pH change in the laminated sheet of Example 48 after the storage based on the measurement method 2.

### DETAILED DESCRIPTION OF THE INVENTION

The tooth whitening patch or the multi-layer strip described in the above-mentioned Patent Documents 1 and 2 uses peroxides such as hydrogen peroxide as a whitening component, and thus may give excessive stimulus to the tooth surfaces or the mucous membrane in the oral cavity, and cause hyperesthesia and the like, and may not exert enough effect against tooth discoloration due to aging and the like. Furthermore, the method of producing a laminate described in Patent Document 3 does not disclose specific components or pH to exert the whitening effect when the laminate is attached to teeth, and enough study for further enhancing the safety and improving the tooth whitening effect.

Consequently, the present invention relates to an adhesive sheet for teeth, which has high safety, and makes it possible to obtain an excellent tooth whitening effect by a simple means of just being attached to teeth.

The present inventors studied substances that can be a factor for stains of teeth by aging, and found that a component in minute solid matter having a thickness of less than several μm adhered to the tooth surface is a factor for the stains. The present inventors further analyzed this minute solid matter in detail, and found that not only inorganic substances such as calcium phosphates but also organic substances are contained in this minute solid matter, and there are many spots in which these inorganic substances and organic substances are not uniformly mixed, but unevenly distributed respectively to form the solid matter. Now, the present inventors found that unexpectedly an excellent effect of removing the minute solid matter on the tooth surface having the organic substances and the inorganic substances unevenly distributed, is exerted by giving time-dependent change of the pH environment of the tooth surface and allowing specific components to act on the minute solid matter, and found that an adhesive sheet for teeth having a layer having weak alkaline when the layer is dissolved in water and a layer containing specific components and having weak acidic when the layer is dissolved in water can exert an excellent whitening effect when the adhesive sheet is attached to teeth.

The adhesive sheet for teeth of the present invention has high safety and a good feeling upon use, and can exert an excellent whitening effect and make teeth white and glossy by sufficiently allowing specific components to act when the adhesive sheet is attached to teeth and time-dependently changing the pH environment of the tooth surface.

Hereinafter, the present invention will be explained in detail.

The minute stain or solid matter adhered or deposited on the tooth surface in the present invention refers to a solid matter being produced on the tooth surface and having a thickness of less than several μm, and such solid matter includes aggregates containing inorganic substances as a main component, and aggregates containing organic substances as a main component, and also includes those having a complex structure that presents a layer shape or see-island shape in which both of them are unevenly distributed. This minute solid matter is considered to be mainly formed by adhesion of components such as a protein, calcium and phosphorus in the saliva, and formed by slow deposition of the adhered components, and is easily produced by promotion of the adhesion or deposition by deterioration of the environment in the oral cavity due to aging or a decrease of saliva secretion, and the like, and is difficult to remove with an ordinary brushing treatment.

The adhesive sheet of the present invention has a layer (A) that has a pH of 6.6 or more and 10.5 or less when the layer (A) is dissolved in water, and a layer (B) that comprises a component (b) of one more kinds selected from phytic acid, hexamethaphosphoric acid, an organic acid having a pKa of from 4.5 to 7.0 at 25° C. and salts thereof, and that has a pH of 3.5 or more and 6.5 or less when the layer (B) is dissolved in water. By the adhesive sheet having the layer (A) that has a pH of 6.6 or more and 10.5 or less when the layer (A) is dissolved in water, and the layer (B) that comprises the component (b), and that has a pH of 3.5 or more and 6.5 or less when the layer (B) is dissolved in water, the layer (A) and the layer (B) can be sequentially dissolved on the tooth surfaces when the adhesive sheet is attached to teeth, and the adhesive sheet can release the component (b) comprised in the layer (B) to the tooth surfaces while time-dependently changing the pH environment of the tooth surface. By giving time-dependent change of the pH environment to the tooth surface, the adhesive sheet can exert unexpectedly an excellent effect of removing minute solid matters adhered or deposited on teeth in which organic substances or inorganic substances are localized, and further effectively exert a tooth whitening action by the component (b), and make teeth white and glossy.

The layer (A) contained in the adhesive sheet of the present invention has a pH of 6.6 or more and 10.5 or less when the layer (A) is dissolved in water. By adjusting the pH of the layer (A) to 6.6 or more and 10.5 or less when the layer (A) is dissolved in water as described above, the adhesive sheet of the present invention can be dissolved in saliva and the like and render the pH environment of the tooth surface to be 6.6 or more and 10.5 or less when the adhesive sheet is attached to teeth, and is allowed to particularly effectively act on organic stains in the solid matter whereby to render inorganic stains in the solid matter easily removable, and render the component (b) to easily act under weak acidic environment and remove low crystalline apatite selectively and favorably whereby to sufficiently exert an immediately effective gloss imparting effect. The pH of the layer (A) when the layer (A) is dissolved in water is a value at room temperature (25° C.), and is 6.6 or more, preferably 7.1 or more, even more preferably 7.5 or more, even more preferably 8 or more from the viewpoint of rendering the organic matter stains easily removable and showing the gloss imparting effect and the whitening effect, and is 10.5 or less, preferably 10 or less, even more preferably 9.5 or less, even more preferably 9 or less from the viewpoint of the action to the mucous membrane in the oral cavity. In addition, the pH of the layer (A) is from 6.6 to 10.5, preferably from 7.1 to from 10, even more preferably from 7.5 to 9.5, even more preferably from 8 to 9 when the layer (A) is dissolved in water.

Furthermore, as the measurement method for the pH of the layer (A), the pH of the layer (A) is measured by applying a minimum amount of water allowing pH measurement onto the layer (A). Specifically, an amount of water allowing dissolution of the layer (A), for example, 0.1 to 0.5 g of water, is dropped at room temperature (25° C.) on the adhesive sheet of the present invention that is inclined by 45 degrees from the horizontal surface with the layer (A) upward, then the dropped water is rapidly collected, and the pH is measured by for example, a compact pH meter B-712 (HORIBA, Ltd.) and the like. Furthermore, the water dissolving the layer (A) is purified water, and also means to include distilled water or deionized water.

In adjustment of the pH of the layer (A) to the above-described range, a pH regulator is preferably contained. As such pH regulator, one kind alone or a combination of two or more kinds may be used in a range in which the effect of rendering the minute solid matter easily removable and favorably removing the solid matter by the layer (B), the effect of enhancing the gloss-imparting and the whitening effect are not inhibited, and decalcification of teeth can be suppressed.

In addition, a gelling agent is preferably further contained in the layer (A) contained in the adhesive sheet of the present invention. Examples of such gelling agent include one or more selected from pullulan, sodium carboxymethylcellulose, hydroxyethylcellulose, a carboxyvinyl polymer, xanthan gum, carrageenan, sodium alginate, hydroxypropylcellulose, hydroxypropylmethylcellulose, guar gum and sodium chondroitin sulfate. Among them, one or two or more non-ionic polymers such as pullulan, hydroxyethylcellulose, hydroxypropylmethylcellulose, guar gum, hydroxypropylcellulose are preferably contained from the viewpoint of good producibility of the sheet and good controllability of the solubility of the layer (A), and one or more selected from pullulan, hydroxyethylcellulose, hydroxypropylmethylcellulose and hydroxypropylcellulose are more preferably contained from the viewpoint of the immediately effective gloss imparting effect and the whitening effect.

In addition, a component (a) of one or more kinds selected from alkyl sulfuric acid, acylmethyltauric acid, N-acylsarcosic acid, orthophosphoric acid, condensed phosphoric acid and salts thereof is preferably further contained in the layer (A) contained in the adhesive sheet of the present invention from the viewpoint of sufficiently exerting the tooth whitening action with the pH environment change and the action of the component (b) by releasing the component (b) contained in the layer (B) to the oral cavity while changing the pH environment in the oral cavity. Such component (a) is effective as an enhancing agent for the gloss-imparting action by the component (b) contained in the layer (B) described below, but has a problem that the component (a) and the component (b) cannot be treated at a pH that is suitable for the respective actions of the component (a) and the component (b) if the component (a) and the component (b) are applied at the same time. However, according to the adhesive sheet of the present invention, it is possible to allow the layer (A) and the layer (B) act in order when the adhesive sheet is attached to teeth, make the component (a) effectively act as an enhancing agent for the gloss-imparting action by the component (b), and enhance the tooth whitening action and the gloss-imparting action.

Examples of the alkyl sulfuric acid, acylmethyltauric acid, N-acylsarcosic acid and salts thereof of the component (a) include laurylsulfuric acid, lauroylmethyltauric acid, N-lauroylsarcosic acid and salts thereof. Among them, the salts thereof are preferable, and alkali metal salts such as sodium salts and potassium salts are more preferable. Specifically, examples of the salts thereof include sodium laurylsulfate, potassium laurylsulfate, sodium lauroylmethyltaurate, potassium lauroylmethyltaurate, sodium lauroylsarcosinate and potassium lauroylsarcosinate. In addition, examples of the orthophosphoric acid and condensed phosphoric acid include pyrophosphoric acid, tripolyphosphoric acid, tetrapolyphosphoric acid and methaphosphoric acid. The salts thereof are preferably alkali metal salts such as sodium salts and potassium salts. Specifically, examples of the salts thereof include, for example, sodium pyrophosphate, potassium pyrophosphate, sodium tripolyphosphate and potassium tripolyphosphate. Among them, the salts of pyrophosphoric acid and tripolyphosphoric acid are preferred, and sodium pyrophosphate or sodium tripolyphosphate are more preferred from the viewpoint of enhancing the tooth whitening action and the gloss-imparting action with short term use.

The content of the component (a) in the layer (A) is preferably 1% by mass or more, more preferably 2% by mass or more, even more preferably 3% by mass or more based on dry mass from the viewpoint of sufficiently exerting the effect of rendering the minute solid matter easily removable and favorably removing this, the effect of enhancing the gloss-imparting and the whitening effect. The content of the component (a) in the layer (A) is preferably 15% by mass or less, more preferably 10% by mass or less, even more preferably 8.5% by mass or less, even more preferably 7% by mass or less based on dry mass from the viewpoint of a feeling upon use such as taste and friction. In addition, the content of the component (a) in the layer (A) is preferably from 1 to 15% by mass, more preferably from 2 to 10% by mass, even more preferably from 3 to 8.5% by mass, even more preferably from 3 to 7% by mass based on dry mass. Furthermore, the content of the component (a) in the layer (A) is a value obtained by converting the total amount to the amount of the acid (the amount in terms of the acid).

The thickness of the layer (A) is preferably 100 μm or less, more preferably 50 μm or less, even more preferably 20 μm or less from the viewpoint of dissolving the layer (A) within a suitable time and securing suitable flexibility as a sheet. The thickness of the layer (A) is preferably 1 μm or more, more preferably 5 μm or more, even more preferably 7 μm or more from the viewpoint of allowing the tooth whitening action and enhancement of the gloss-imparting action. The thickness of the layer (A) is preferably from 1 to 100 μm, more preferably from 5 to 50 μm, even more preferably from 7 to 20 μm.

The layer (A) is preferably formed using a base material solution (A). The layer (A) can be formed by layering the base material solution (A), and then subjecting it to dry process. When the component (a) is contained in such base material solution (A), the content of the component (a) in the base material solution (A) is preferably 0.025% by mass or more, more preferably 0.125% by mass or more, even more preferably 0.25% by mass or more from the viewpoint of sufficiently exerting the effect of rendering the minute solid matter easily removable and favorably removing this, the effect of enhancing the gloss-imparting and the whitening effect. The content of the component (a) in the base material solution (A) is preferably 7.5% by mass or less, more preferably 5% by mass or less, even more preferably 2.5% by mass or less from the viewpoint of a feeling upon use such as taste and friction. In addition, the content of the component (a) in the base material solution (A) is preferably 0.025% by mass or more and 7.5% or less by mass or less, more preferably 0.125 to 5% by mass, even more preferably 0.25 to 2.5% by mass. Furthermore, the content of the component (a) in the base material solution (A) is a value obtained by measuring a total amount by neutralization using potassium hydroxide or sodium hydroxide, and converting the total amount to the amount in terms of the acid.

The pH of the base material solution (A) is preferably 6.6 or more, more preferably 7.1 or more, even more preferably 7.5 or more, even more preferably 8 or more from the viewpoint of realizing the above-mentioned pH when the layer (A) is dissolved in water, and is 10.5 or less, more preferably 10 or less, even more preferably 9.5 or less, even more preferably 9 or less from the viewpoint of the action to the mucous membrane in the oral cavity. The pH of the base material solution (A) is preferably from 6.6 to 10.5, more preferably from 7.1 to 10, even more preferably from 7.5 to 9.5 or less, even more preferably 8 to 9 from the similar viewpoints.

It is preferred that one or more non-ionic polymers such as pullulan, hydroxyethylcellulose, guar gum, hydroxypropylcellulose and hydroxypropylmethylcellulose are contained as a gelling agent from the viewpoint of film formability when the base material solution (A) has a high salt concentration in formation of the layer (A) using the base material solution (A).

A solvent such as water and/or ethanol may be contained in an amount of about from 1 to 20 folds in the layer (A) or the base material solution (A) in addition to the above components. It is especially preferred that the base material solution (A) forms slurry using a solvent such as water and/or ethanol. The layer (A) is formed by layering the base material solution (A) in the slurry form using a solvent such as water and/or ethanol, and then subjecting it to dry process. The water amount in the layer (A) is preferably 18 mass parts or less, more preferably 15 mass parts or less, even more preferably 14 mass parts or less relative to 100 mass parts by dry weight of the layer (A), and from the viewpoint of flexibility, adhesion and manufacturability of the sheet, the water amount in the layer (A) is preferably 2 mass parts or more, more preferably 3 mass parts or more, even more preferably 5 mass parts or more.

The layer (B) in the adhesive sheet of the present invention contains a component (b) of one or more kinds selected from phytic acid, hexamethaphosphoric acid, an organic acid having a pKa of from 4.5 to 7.0 at 25° C. and salts thereof. Such component (b) has excellent tooth whitening action and gloss-imparting action in an acidic to weak acidic range, which allows the layer (A) and the layer (B) act on teeth in order, whereby to remarkably enhance the gloss-imparting action, and obtain excellent gloss imparting effect on teeth and tooth whitening effect even with short term use.

Examples of the organic acid having a pKa of from 4.5 to 7.0 at 25° C. include fumaric acid, malic acid, tartaric acid, citric acid, glutaric acid and the like. Among them, the component (b) is preferably phytic acid or a salt thereof from the viewpoint of exerting excellent tooth whitening action and gloss-imparting action. Furthermore, the pKa at 25° C. is a reciprocal number of logarithm of the acid dissociation constant (for example, “Handbooks of Chemistry-Fundamentals, Vol. 2”, page 993, 6^{th} expression published by MARUZEN Co., Ltd. on Sep. 20, 1981; “Handbooks of Chemistry-Fundamentals, Vol. 4”, page 317, published by MARUZEN Co., Ltd. on Sep. 30, 1993, and the like), and means a value measured for 25° C. water solution.

The salts of these phytic acid, hexamethaphosphoric acid, and an organic acid having a pKa of from 4.5 to 7.0 at 25° C. are preferably alkali metal salts such as sodium salts and potassium salts.

The layer (B) has a pH of more than 3.5 and 6.5 less than when the layer (B) is dissolved in water. By adjusting the pH of the layer (B) when the layer (B) is dissolved in water to pH 3.5 and more and 6.5 or less as described above, the adhesive sheet of the present invention can be dissolved in saliva and the like and render the pH environment of the tooth surface to be pH 3.5 or more and 6.5 or less while releasing the component (b) when the adhesive sheet of the present invention is attached to teeth, and selectively remove particularly low crystalline apatite at from the tooth surfaces to the interprismic space of the enamel, whereby to effectively act on inorganic stains to remove the minute solid matter, and suppress decalcification of tooth surfaces to sufficiently exert the immediately effective gloss imparting effect and the whitening effect. When the layer (B) is dissolved in water, the pH of the layer (B) is a value at 25° C., and is 3.5 or more, more preferably 3.7 or more, even more preferably 4.0 or more, even more preferably 4.2 or more from the viewpoint of suppressing decalcification of tooth surfaces and showing the immediately effective gloss imparting effect and the whitening effect, and is 6.5 or less, more preferably 6.0 or less, even more further preferably 5.5 or less, even more preferably 5.0 or less from the viewpoint of sufficiently showing the gloss imparting effect and the whitening effect by removing the solid matter. In addition, when the layer (B) is dissolved in water, the pH of the layer (B) is from 3.5 to 6.5, preferably from 3.7 to 6.0, more preferably from 4.0 to 5.5 even more preferably from 4.2 to 5.0.

Furthermore, a measurement method for the pH of the layer (B) is such that the pH of the layer (B) is measured by applying a minimum amount of water allowing pH measurement onto the layer (B) at room temperature (25° C.) similarly to the pH measurement method for the layer (A). Specifically, an amount of water allowing dissolution of the layer (B), for example, 0.1 to 0.5 g of water, is dropped on the adhesive sheet of the present invention that is inclined by 45 degrees from the horizontal surface with the layer (B) upward, then the dropped water is rapidly collected, and the pH is measured by, for example, a compact pH meter B-712 (HORIBA, Ltd.) and the like. Furthermore, the water dissolving the layer (B) is purified water, and also means to include distilled water or deionized water.

The content of the component (b) in the layer (B) is preferably 5% by mass or more, more preferably 10% by mass or more, even more preferably 12% by mass or more based on dry mass from the viewpoint of sufficiently exerting the minute solid matter-removing effect and the gloss imparting effect. The content of the component (b) in the layer (B) is preferably 30% by mass or less, more preferably 28% by mass or less based on dry mass from the viewpoint of suppressing decalcification of teeth and from the viewpoint of taste and friction, and further preferably 20% by mass or less from the viewpoint of manufacturability. In addition, the content of the component (b) in the layer (B) is preferably from 5 to 30% by mass, and more preferably from 10 to 28% by mass, even more preferably from 12 to 28% by mass based on dry mass, and the content of the component (b) in the layer (B) is still further preferably from 12 to 20% by mass or more from the viewpoint of manufacturability. Furthermore, the content of the component (b) in the layer (B) is a value obtained by converting the total amount to the amount of the acid (amount in terms of the acid).

In adjustment of the pH of the layer (B) to the described-above range, a pH regulator is preferably contained. Examples of the pH regulator include inorganic acids such as hydrochloric acid and sulfuric acid, hydroxides such as sodium hydroxide, ammonia or ammonia water, lower alkanol amines, basic amino acids such as arginine and lysine, and the like in a range in which the gloss imparting effect and the whitening effect by the component (b) are not inhibited, and decalcification of teeth can be suppressed. These may be used individually or two or more may be used in combination. Among them, hydroxides such as sodium hydroxide are preferable.

In addition, a gelling agent is preferably further contained in the layer (B) contained in the adhesive sheet of the present invention. Examples of such gelling agent include one or two or more selected from pullulan, sodium carboxymethylcellulose, hydroxyethylcellulose, a carboxyvinyl polymer, xanthan gum, carrageenan, sodium alginate, hydroxypropylcellulose, hydroxypropylmethylcellulose, guar gum and sodium chondroitin sulfate. Among them, one or more non-ionic polymers such as pullulan, hydroxyethylcellulose, guar gum, hydroxypropylcellulose and hydroxypropylmethylcellulose are preferably contained from the viewpoint of good producibility of the sheet and the like, and one or more selected from pullulan, hydroxyethylcellulose, hydroxypropylmethylcellulose and hydroxypropylcellulose are more preferably contained from the viewpoint of the immediately effective gloss imparting effect and the whitening effect.

The thickness of the layer (B) is preferably 200 μm or less, more preferably 100 μm or less, even more preferably 60 μm or less from the viewpoint of dissolving the layer within a suitable time and securing suitable flexibility as a sheet. The thickness of the layer (B) is preferably 5 μm or more, more preferably 10 μm or more, even more preferably 15 μm or more, even more preferably 20 μm or more from the viewpoint of allowing the tooth whitening action and enhancement of the gloss-imparting action with short term use. The thickness of the layer (B) is preferably from 5 to 200 μm, more preferably from 10 to 100 μm, even more preferably from 15 to 60 μm, even more preferably from 20 to 60 μm.

The layer (B) is preferably formed using a base material solution (B) containing the component (b). The layer (B) can be formed by layering the base material solution (B), and then subjecting it to dry process. The content of the component (b) in such base material solution (B) is preferably 0.25% by mass or more, more preferably 1.25% by mass or more, even more preferably 2.5% by mass or more from the viewpoint of sufficiently exerting the minute solid matter-removing effect and the gloss imparting effect. The content of the component (b) in the base material solution (B) is preferably 12.5% by mass or less, more preferably 10% by mass or less and further preferably 7.5% by mass or less from the viewpoint of suppressing decalcification of teeth, and from the viewpoint of taste and friction. In addition, the content of the component (b) in the base material solution (B) is preferably from 0.25 to 12.5% by mass or less, more preferably from 1.25 to 10% by mass, even more preferably from 2.5 to 7.5% by mass. Furthermore, the content of the component (b) in the base material solution (B) is a value obtained by measuring a total amount by neutralization using potassium hydroxide or sodium hydroxide, and converting the total amount to the amount of the acid.

The pH of the base material solution (B) is preferably 3.5 or more, more preferably 3.7 or more, even more preferably 4.0 or more, even more preferably 4.2 or more from the viewpoint of realizing the above-mentioned pH when the layer (B) is dissolved in water. The pH of the base material solution (B) is preferably 6.5 or less, even more preferably 6.0 or less, even more preferably 5.5 or less, even more preferably 5.0 or less from the similar viewpoints. Furthermore, the pH of the base material solution is also a value at 25° C.

It is preferred that one or more non-ionic polymers such as pullulan, hydroxyethylcellulose, guar gum, hydroxypropylcellulose and hydroxypropylmethylcellulose are contained as a gelling agent from the viewpoint of film formability when the base material solution (A) has a high salt concentration in formation of the layer (B) using the base material solution (B), similarly to formation of the layer (A).

Similarly to the layer (A) or the base material solution (A), a solvent such as water and/or ethanol may be contained in an amount of about from 1 to 20 folds in the layer (B) or the base material solution (B) in addition to the above components. It is especially preferred that the base material solution (B) forms slurry using a solvent such as water and/or ethanol. The layer (B) is formed by layering the base material solution (B) in the slurry form using a solvent such as water and/or ethanol, and then subjecting it to dry process. The water amount in the layer (B) is preferably 18 mass parts or less, more preferably 15 mass parts or less, even more preferably 14 mass parts or less relative to 100 mass parts by dry weight of the layer (B), and from the viewpoint of flexibility, adhesion and manufacturability of the sheet, the water amount in the layer (B) is preferably 2 mass parts or more, more preferably 3 mass parts or more, even more preferably 5 mass parts or more.

The content of polyvalent cations in the layer (B) is preferably suppressed from the viewpoint of preventing the component (b) from being insoluble, and preventing decrease of the solid matter-removing effect. The content of polyvalent cation is measured by ICP emission spectrometry (ICP emission spectrometer: Optima 5300DV manufactured by PerkinElmer Inc.). The total content of polyvalent cation is less than 0.1-fold mol, more preferably 0.02-fold mol or less, even more preferably 0.01-fold mol or less relative to the component (b). That is, it is desired that a cationic antibacterial agent, a cationic surfactant or an agent for mainly supplying polyvalent cation such as aluminum, calcium, magnesium, iron, zinc and tin is not blended in the layer (B). Alternatively, the layer (B) does not contain polyvalent cations except the case of inevitable contamination.

In addition, absorbents such as zeolite and activated carbon are likely to reduce the solid matter-removing effect of the component (b), and thus the content of such absorbents is preferably less than 0.005% by mass, even more preferably 0.0005% by mass or less based on dry mass in the layer (B), and preferably the absorbent is not contained except the case of inevitable contamination.

Either of the layer (A) and the layer (B) may be first dissolved when the adhesive sheet for teeth of the present invention is attached to teeth. That is, the layer constituting the side pasted to teeth may be the layer (A), or may be the layer (B). Among them, the layer constituting the side attached to teeth is preferably the layer (A) from the viewpoint of first rendering the tooth surfaces to be under the environment of pH 6.6 or more and 10.5 or less and then allowing the component (b) to sufficiently act under the environment of pH 3.5 or more and 6.5 or less when the adhesive sheet of the present invention is attached to teeth. In addition, also from the viewpoint of allowing first the component (a) to sufficiently act under the environment of pH 6.6 or more and 10.5 or less and then allowing to sufficiently act the component (b) under the environment of pH 3.5 or more and 6.5 or less when the adhesive sheet of the present invention is pasted to teeth, the layer constituting the side attached to teeth is preferably the layer (A). In this case, the pH is preferably adjusted in consideration that the layer (B) is dissolved while being pasted to the layer (A), and buffered to the pH environment of the layer (A). In addition, the layer constituting the side attached to teeth is preferably the layer (B) from the viewpoint of first allowing the component (b) to sufficiently act under the environment of pH 3.5 or more and 6.5 or less, and then allowing the component (a) to sufficiently act under the environment of pH 6.6 or more and 10.5 or less when the adhesive sheet of the present invention is attached to teeth. In this case, the pH of the layer (B) is preferably adjusted such that the component (b) acts under the environment of pH 5.5 or more and 6.5 or less in order to prevent decalcification of the enamel of the tooth surface. That is, the pH of the layer (B) is preferably from 5.5 to 6.5 when the layer (B) is dissolved in water.

Furthermore, the thickness ratio between the layer (A) and the layer (B) (Aₜ:Bₜ) is preferably from 1:1 to 1:10, more preferably from 1:2 to 1:9, even more preferably from 1:3 to 1:8.

The layer (A) and the layer (B) preferably maintain the performance of bringing different pH environments from each other when the layer (A) and the layer (B) are dissolved in water from the production to the storage from the point of showing the tooth whitening effect by bringing different pH environments from each other when the layer (A) and the layer (B) are dissolved in water. The layer (A) and the layer (B) are water-soluble as described above, and thus from the viewpoint of preventing each component contained in one layer from moving to the other layers, and securing flexibility or usability of the sheet, the water content in the adhesive sheet for teeth of the present invention is preferably 18 mass parts or less, more preferably 15 mass parts or less, even more preferably 14 mass parts or less, and from the viewpoint of securing flexibility, adhesion and manufacturability of the sheet, the water content in the adhesive sheet for teeth of the present invention is preferably 2 mass parts or more, more preferably 3 mass parts or more, even more preferably 5 mass parts or more relative to 100 mass parts by dry mass of the adhesive sheet for teeth.

Furthermore, the solubility of the layer (B) in water is preferably lower than the solubility of the layer (A) in water. For example, by forming the layer (A) containing pullulan as the gelling agent, and forming the layer (B) containing hydroxymethylpropylcellulose as the gelling agent, it is possible to obtain an adhesive sheet for teeth that has high adhesion to teeth, and yet has suitable stiffness, and can be pasted even with wet hands. Furthermore, for example, when the adhesive sheet is once attached to teeth, but is desired to change the position to be attached, the adhesive sheet can be peeled and re-pasted again.

The adhesive sheet of the present invention preferably has a layer (C) that does not collapse during dissolution of the layer (A) and/or the layer (B) as a layer other than the layer (A) and the layer (B). Among them, the layer (C) is preferably a layer constituting the opposite side to the side attached to teeth. By the layer (C) constituting the opposite side to the side attached to teeth, it is possible to effectively prevent the layer contained in the sheet from being dissolved from the opposite side to the side pasted to teeth in saliva and the like and to preferentially dissolve the layer contained in the sheet from the side attached to teeth, when the adhesive sheet of the present invention is attached to teeth. The layer (C) that does not collapse during dissolution of the layer (A) and/or the layer (B) is formed by containing, for example, a component (c) that is a sparingly water-soluble substance. That is, the sparingly water-soluble substance in the present invention is a substance rendering the layer (C) not to collapse during dissolution of the layer (A) and/or the layer (B), and preferably to collapse after dissolution of the layer (A) and/or the layer (B). Furthermore, “not collapsing” means no exposure of the layer (A) and/or the layer (B) due to progress of the collapsing. Examples of the component (c) include specifically, one or more (co)polymers selected from cellulose acetate phthalate, polyvinyl acetate, ethylcellulose, polymethylmethacrylate, a methacryloylethylbetaine-methacrylate copolymer, a methacrylic acid copolymer and an aminoalkyl methacrylate copolymer. Among them, ethylcellulose or cellulose acetate phthalate is preferable from the viewpoint of enhancing followability of the layer (C) to the layer (A) and the layer (B), and from the viewpoint of the immediately effective gloss-imparting effect, the whitening effect and the like.

The content of the component (c) in the layer (C) is preferably 40% by mass or more, more preferably 50% by mass or more, even more preferably 60% by mass or more based on dry mass from the viewpoint of effectively preventing dissolution of the layer from the opposite side to the side attached to teeth, whereby to preferentially dissolve the layer constituting the side attached to teeth. The content of the component (c) in the layer (C) is preferably 100% by mass or less, more preferably 90% by mass or less, even more preferably 80% by mass or less based on dry mass from the viewpoint of ease use for a user. In addition, the content of the component (c) in the layer (C) is preferably form 40 to 100% by mass, more preferably from 50 to 90% by mass based on dry mass.

The thickness of the layer (C) is preferably 0.5 μm or more, more preferably 1 μm or more from the viewpoint of effectively preventing dissolution of the layer from the opposite side to the side attached to teeth, whereby to preferentially dissolve the layer constituting the side attached to teeth. The thickness of the layer (C) is preferably 40 μm or less, more preferably 10 μm or less from the viewpoint of ease use for a user.

The adhesive sheet of the present invention may further have a middle layer (X) in addition to the layer (A), the layer (B) and the layer (C). Such middle layer (X) may be formed between any layers of the layer (A), the layer (B) and the layer (C). For example, the adhesive sheet of the present invention has the middle layer (X) between the layer (A) and the layer (B), whereby to easily form these layers in a separate state without mixing of the layer (A) and the layer (B) at the time of manufacture, and easily improve the pH stability of the layer (A) and the layer (B) at the time of storage, and make it easy to release the components dissolved and contained in one layer into the oral cavity, and then subsequently release the components dissolved and contained in the other layers into the oral cavity at the time of use.

The material of the middle layer (X) is preferably a non-ionic polymer such as pullulan, hydroxyethylcellulose, guar gum and hydroxypropylcellulose, or the like from the viewpoint of enhancing followability to the other layers, and is preferably one or two or more (co)polymers selected from cellulose acetate phthalate, polyvinyl acetate, ethylcellulose, polymethylmethacrylate, a methacryloylethylbetaine-methacrylate copolymer, a methacrylic acid copolymer and an aminoalkyl methacrylate copolymer, and shellac from the viewpoint of enhancing separation of the layer (A) and the layer (B). The material of the middle layer (X) is preferably shellac from the viewpoint of the pH stability when the layer (A) and the layer (B) are dissolved in water. The water content in the middle layer (X) is preferably 18 mass parts or less, more preferably 15 mass parts or less, even more preferably 14 mass parts or less relative to 100 mass parts by dry weight of the middle layer (X), and is preferably 2 mass parts or more, more preferably 3 mass parts or more, even more preferably 5 mass parts or more from the viewpoint of the flexibility, the adhesion and the manufacturability of the adhesive sheet.

The thickness of the middle layer (X) is, for example, preferably 1 μm or more, more preferably 5 μm or more, even more preferably 10 μm or more from the viewpoint of improving separation of the layer (A) and the layer (B) and the pH stability of the layer (A) and the layer (B) when a non-ionic polymer such as pullulan is used. The thickness of the middle layer (X) is preferably 100 μm or less, more preferably 50 μm or less, even more preferably 30 μm or less from the viewpoint of ease use for a user. On the other hand, the thickness of the middle layer (X) is preferably 0.1 μm or more, more preferably 0.5 μm or more, even more preferably 1 μm or more when ethylcellulose, shellac or the like is used. In addition, the thickness of the middle layer (X) is preferably 5 μm or less, more preferably 3 μm or less from the viewpoint of ease use for a user.

The middle layer (X) preferably has a pH of from 6.5 to 7.0.

The total thickness of the adhesive sheet of the present invention is preferably 10 μm or more, more preferably 20 μm or more, even more preferably 30 μm or more, and is preferably 1000 μm or less, more preferably 500 μm or less and further preferably 100 μm or less from the viewpoint of enhancing followability to teeth when the adhesive sheet is attached, and from the viewpoint of showing the immediately effective gloss-imparting effect and the whitening effect.

The adhesive sheet of the present invention may further contain properly other components such as a sweetening agent, a preservative, an antibacterial agent, a medicinally active ingredient, a pigment, a colorant and a flavor. These other components may be contained in an appropriate layer of the layer (A), the layer (B), and the layer (C) and the middle layer (X) as necessary as long as the effects of the present invention are not harmed.

When the layer (C) or the middle layer (X) is used in the adhesive sheet of the present invention as a layer other than the layer (A) and the layer (B), a plasticizer is preferably contained from the viewpoint of imparting suitable stiffness or flexibility, and from the viewpoint of securing good film formability in these layers. Examples of such plasticizer include, for example, sugar alcohols such as sorbitol, erythritol, mannitol, maltitol and lactitol, polyvalent alcohols such as glycerol, propylene glycol, and polyethylene glycol, and the like. One kind of them may be used alone, or two or more kinds in combination may be used.

The content of the plasticizer is preferably 0.01% by mass or more, more preferably 0.1% by mass or more, even more preferably 1% by mass or more based on dry mass in each layer. The content of the plasticizer is preferably 50% by mass or less, more preferably 40% by mass or less, even more preferably 30% by mass or less based on dry mass in each layer.

In production of the adhesive sheet of the present invention, the adhesive sheet of the present invention may be produced by forming each of sheets including single layers of the layer (A) and the layer (B), and the layer (C) and the middle layer (X) as necessary, and then laminating the sheets, or laminating the sheets sequentially on a substrate. For example, when a sheet including single layer of the layer (A) is formed, a base material solution (A) containing the component (a) is produced, and then the base material solution (A) is coated on a polyethylene terephthalate (PET) film subjected to mold release treatment and the like in consideration of the thickness, and dried. Then, the PET film is detached, whereby to obtain a sheet including respective single layers. Meanwhile, a sheet constituting the side attached to teeth may be supported as it is without detachment of the PET film. Then, the PET film is arranged at the bottom layer, and a sheet including single layer constituting the side attached to teeth and sheets including the other single layers are sequentially laminated thereon, and then pressed using, for example, a heated pressure device. Then, the PET film at the bottom layer is detached, whereby to obtain the adhesive sheet of the present invention.

In addition, when the sheets are sequentially laminated on the substrate, for example, the base material solution (A) is first coated on a PET film as a substrate, and dried whereby to form the layer (A) when the layer (A) constitutes the side attached to teeth. Then, the middle layer (X) is formed as necessary on the layer (A), and the base material solution (B) is coated and dried to form the layer (B). Then, the sheet is pressed using a heated pressure device, whereby to obtain the adhesive sheet of the present invention.

Furthermore, the adhesive sheet for teeth of the present invention is preferably sealed in a water-barrier packaging bag from the viewpoint of maintaining suitable water content of the adhesive sheet for teeth of the present invention. Examples of such packaging bag include, for example, a packaging bag that is a resin sheet of multi-layer structure in which the innermost layer is polyethylene, and three sides or four sides of the sheet are heat-sealed. For example, a form of the packaging bag such as a pillow bag and a gusseted bag may be used. In addition, the resin sheet of multi-layer structure constituting such packaging bag is preferably provided with an aluminum layer in the middle layer from the viewpoint of securing a high water barrier property.

By just being attached to teeth, the adhesive sheet of the present invention can bring excellent gloss imparting effect and whitening effect to a tooth that is the attached. The time from immediately after attaching to teeth until entirely dissolving the layer (A) and the layer (B) in water is preferably 90 minutes or less, more preferably 60 minutes or less, even more preferably 30 minutes or less from the viewpoint of maintaining the concentrations of the component (a) and the component (b) released to the tooth surfaces and from the viewpoint of ease use for a user when the adhesive sheet is attached to teeth. The time is preferably 1 minute or more, more preferably 5 minutes or more, even more preferably 10 minutes or more from the viewpoint of exerting the gloss imparting effect and the whitening effect effectively when the adhesive sheet of the present invention is attached to teeth.

The adhesive sheet for teeth of the present invention is attached to teeth at a proper interval, specifically at an interval of preferably from 1 to 24 hours, more preferably from 2 to 12 hours, preferably twice or more, more preferably 4 times or more, even more preferably 6 times or more, i.e., the adhesive sheet for teeth of the present invention is used repeatedly, which makes it possible to further exert the effects of the present invention, suppress damage to the enamel surfaces of the teeth, and obtain teeth having further natural luster or gloss.

The time until entire dissolution of the layer (A) in water (dissolution time) is preferably 15 minutes or less, more preferably 10 minutes or less, even more preferably 5 minute or less from viewpoint of maintaining the concentration of the component (a) released to the tooth surfaces and from the viewpoint of ease use for a user. The dissolution time of the layer (A) is preferably 0.1 minutes or more, more preferably 0.3 minutes or more, even more preferably 0.5 minutes or more from the viewpoint of effectively exerting the gloss imparting effect and the whitening effect when the adhesive sheet of the present invention is attached to teeth.

The time until entire dissolution of the layer (B) in water (dissolution time) is preferably 75 minute or less, more preferably 50 minutes or less, even more preferably 30 minutes or less, even more preferably 25 minute or less, even more preferably 15 minute or less from the viewpoint of maintaining the concentration of the component (b) released to the tooth surfaces and from the viewpoint of ease use for a user. The dissolution time of the layer (B) is preferably 1 minute or more, more preferably 3 minutes or more, even more preferably 5 minutes or more from the viewpoint of effectively exerting the gloss imparting effect and the whitening effect when the adhesive sheet of the present invention is attached to teeth.

The time until entire dissolution of the layer (B) in water (dissolution time) is preferably longer than the time until entire dissolution of the layer (A) in water (dissolution time). The time ratio until entire dissolution in water between the layer (A) and the layer (B) (dissolution time), (Aₘ:Bₘ), is preferably 1:1 to 1:15, more preferably from 1:1 to 1:10, more preferably from 1:2 to 1:9, even more preferably from 1:3 to 1:8.

Furthermore, in measurement of the time until entire dissolution of each layer in water (dissolution time), for example, in the case of the adhesive sheet in which the layer (A) and the layer (B) are laminated, the time until the components contained in the layer (B) is released after water is applied to the side of the layer (A) at a speed of 0.1 g/min, is assumed as the time until entire dissolution of the layer (A) in water (dissolution time). Then, the time until the amount of the components contained in the layer (B) is not increased in water after the layer (A) is entirely dissolved in water and then water is applied, is assumed as the time until entire dissolution of the layer (B) in water (dissolution time).

In addition, for example, when the layer (A) containing the component (a) and the middle layer (X) are laminated, the time until the amount of the component (a) contained in the layer (A) is not increased in water after water is applied to the side of the layer (A), is assumed as the time until entire dissolution of the layer (A) in water (dissolution time).

In relation to the embodiments mentioned above, the present invention further discloses the following adhesive sheets for teeth.

[1] An adhesive sheet for teeth, which comprises

a layer (A) that has a pH of 6.6 or more and 10.5 or less when the layer (A) is dissolved in water, and

a layer (B) that comprises a component (b) of one or more kinds selected from phytic acid, hexamethaphosphoric acid, an organic acid having a pKa of from 4.5 to 7.0 at 25° C. and salts thereof, and that has a pH of 4 or more and 6.5 or less when the layer (B) is dissolved in water.

[2] The adhesive sheet for teeth of [1] above, wherein the layer (A) comprises a component (a) of one or more kinds selected from alkyl sulfuric acid, acylmethyltauric acid, N-acylsarcosic acid, orthophosphoric acid, condensed phosphoric acid and salts thereof.

[3] The adhesive sheet for teeth of [2] above, wherein the component (a) is one or more selected from sodium laurylsulfate, potassium laurylsulfate, sodium lauroylmethyltaurate, potassium lauroylmethyltaurate, sodium lauroylsarcosinate, potassium lauroylsarcosinate, pyrophosphoric acid, orthophosphoric acid, tripolyphosphoric acid, tetrapolyphosphoric acid, methaphosphoric acid and salts thereof, preferably one or more selected from pyrophosphoric acid, tripolyphosphoric acid and an alkali metal salt thereof, more preferably sodium pyrophosphate.

[4] The adhesive sheet for teeth of any one of [1] to [3] above, wherein when the layer (A) is dissolved in water, the pH of the layer (A) is 7.1 or more, preferably 7.5 or more, more preferably 8 or more, and is 10 or less, preferably 9.5 or less, more preferably 9 or less.

[5] The adhesive sheet for teeth of any one of [1] to [4] above, wherein the thickness of the layer (A) is 100 μm or less, preferably 50 μm or less, more preferably 20 μm or less, and is 1 μm or more, preferably 5 μm or more, more preferably 7 μm or more.

[6] The adhesive sheet for teeth of any one of [1] to [5] above, wherein the layer (A) is formed using a base material solution (A), and the pH of the base material solution (A) is 6.6 or more, preferably 7.1 or more, more preferably 7.5 or more, even more preferably 8 or more, and is 10.5 or less, preferably 10 or less, more preferably 9.5 or less, even more preferably 9 or less.

[7] The adhesive sheet for teeth of any one of [1] to [6] above, wherein the content of the component (a) in the layer (A) is 1% by mass or more, preferably 2% by mass or more, more preferably 3% by mass or more, and is 15% by mass or less, preferably 10% by mass or less, more preferably 8.5% by mass or less, even more preferably 7% by mass or less based on dry mass.

[8] The adhesive sheet for teeth of any one of [1] to [7] above, wherein the content of the component (a) in the base material solution (A) is 0.025% by mass or more, preferably 0.125% by mass or more, more preferably 0.125% by mass or more, and is 7.5% by mass or less, preferably 5% by mass or less, more preferably 2.5% by mass or less.

[9] The adhesive sheet for teeth of any one of [1] to [8] above, wherein the layer (A) further comprises a gelling agent that is one or more selected from pullulan, sodium carboxymethylcellulose, hydroxyethylcellulose, a carboxyvinyl polymer, xanthan gum, carrageenan, sodium alginate, hydroxypropylcellulose, hydroxypropylmethylcellulose, guar gum and sodium chondroitin sulfate.

[10] The adhesive sheet for teeth of any one of [1] to [9] above, wherein the component (b) is one or more selected from phytic acid, fumaric acid, malic acid, tartaric acid, glutaric acid, pyrophosphoric acid, hexamethaphosphoric acid and salts thereof, preferably phytic acid or a salt thereof.

[11] The adhesive sheet for teeth of any one of [1] to [10] above, wherein when the layer (B) is dissolved in water, the pH of the layer (B) is 3.7 or more, preferably 4.0 or more, more preferably 4.2 or more, and is 6.0 or less, preferably 5.5 or less, more preferably 5.0 or less.

[12] The adhesive sheet for teeth of any one of [1] to [11] above, wherein the thickness of the layer (B) is 200 μm or less, preferably 100 μm or less, and 5 μm or more, preferably 10 μm or more, more preferably 15 μm or more, even more preferably 20 μm or more.

[13] The adhesive sheet for teeth of any one of [1] to [12] above, wherein the layer (B) is formed using a base material solution (B), and the pH of the base material solution (B) is 3.5 or more, preferably 3.7 or more, more preferably 4.0 or more, even more preferably 4.2 or more, and is 6.5 or less, preferably 6.0 or less, more preferably 5.5 or less, even more preferably 5.0 or less.

[14] The adhesive sheet for teeth of any one of [1] to [13] above, wherein the content of the component (b) in the layer (B) is 5% by mass or more, preferably 10% by mass or more, more preferably 12% by mass or more, and is 30% by mass or less, preferably 28% by mass or less, more preferably 20% by mass or less based on dry mass.

[15] The adhesive sheet for teeth of any one of [1] to [14] above, wherein the content of the component (b) in the base material solution (B) is 0.25% by mass or more, preferably 1.25% by mass or more, more preferably 2.5% by mass or more, and is 12.5% by mass or less, preferably 10% by mass or less, more preferably 7.5% by mass or less.

[16] The adhesive sheet for teeth of any one of [1] to [15] above, wherein the layer (B) or the base material solution (B) comprises polyvalent cations in a content of less than 0.1-fold mol, preferably 0.02-fold mol or less, more preferably 0.01-fold mol or less relative to the component (b), and even more preferably the layer (B) or the base material solution (B) does not comprise polyvalent cations.

[17] The adhesive sheet for teeth of any one of [1] to [16] above, wherein the layer (B) further comprises a gelling agent that is one or more selected from pullulan, sodium carboxymethylcellulose, hydroxyethylcellulose, a carboxyvinyl polymer, xanthan gum, carrageenan, sodium alginate, hydroxypropylcellulose, hydroxypropylmethylcellulose, guar gum and sodium chondroitin sulfate.

[18] The adhesive sheet for teeth of any one of [1] to [17] above, wherein the thickness ratio between the layer (A) and the layer (B) (Aₜ:Bₜ) is preferably from 1:1 to 1:15, more preferably from 1:1 to 1:10, further preferably from 1:2 to 1:9, more preferably from 1:3 to 1:8.

[19] The adhesive sheet for teeth of any one of [1] to [18] above, wherein the water content in the adhesive sheet for teeth is preferably 18 mass parts or less, more preferably 15 mass parts or less, even more preferably 14 mass parts or less, and is preferably 2 mass parts or more, more preferably 3 mass parts or more, even more preferably 5 mass parts or more relative to 100 mass parts by dry mass of the adhesive sheet for teeth.

[20] The adhesive sheet for teeth of any one of [1] to [19] above, which further comprises a layer (C) that does not collapse during dissolution of the layer (A) and/or the layer (B), and constitutes the opposite side to the side attached to teeth.

[21] The adhesive sheet for teeth of [20] above, wherein the component (c) is one or more (co)polymers selected from cellulose acetate phthalate, polyvinyl acetate, ethylcellulose, polymethylmethacrylate, a methacryloylethylbetaine-methacrylate copolymer, a methacrylic acid copolymer and an aminoalkyl methacrylate copolymer, preferably ethylcellulose or cellulose acetate phthalate.

[22] The adhesive sheet for teeth of any one of [1] to [21] above, wherein the total thickness is 10 μm or more, preferably 20 μm or more, more preferably 30 μm or more, and is 1000 μm or less, preferably 500 μm or less, more preferably 100 μm or less.

[23] The adhesive sheet for teeth of any one of [1] to [22] above, wherein the layer (A) constitutes the side attached to teeth.

[24] The adhesive sheet for teeth of any one of [1] to [22] above, wherein the layer (B) constitutes the side attached to teeth.

[25] The adhesive sheet for teeth of any one of [1] to [24] above, wherein the time from the time immediately after pasting to teeth until entire dissolution of the layer (A) and the layer (B) in water is 90 minutes or less, preferably 60 minutes or less, more preferably 30 minutes or less, and is 1 minute or more, preferably 5 minutes or more, more preferably 10 minutes or more.

[26] The adhesive sheet for teeth of any one of [1] to [25] above, wherein the time ratio until entire dissolution in water between the layer (A) and the layer (B) (Aₘ:Bₘ) is preferably from 1:1 to 1:10, more preferably from 1:2 to 1:9, even more preferably from 1:3 to 1:8.

[27] The adhesive sheet for teeth of any one of [1] to [26] above, which further comprises a middle layer (X).

[28] The adhesive sheet for teeth of [27] above, wherein the material of the middle layer (X) is one or more (co)polymers selected from cellulose acetate phthalate, polyvinyl acetate, ethylcellulose, polymethylmethacrylate, a methacryloylethylbetaine-methacrylate copolymer, a methacrylic acid copolymer and an aminoalkyl methacrylate copolymer, and shellac.

[29] The adhesive sheet for teeth of any one of [1] to [27] above, which is sealed in a packaging bag.

[30] Use of the adhesive sheet for teeth of any one of [1] to [29] above for producing an adhesive sheet for teeth.

[31] The adhesive sheet for teeth of any one of [1] to [29] above for applying the layer (A) and the layer (B) to teeth by applying the layers to teeth and dissolving the layers in saliva.

[32] Use of the adhesive sheet for teeth of any one of [1] to [29] above for whitening teeth.

[33] Use of the adhesive sheet for teeth of any one of [1] to [29] above for imparting gloss to teeth.

### EXAMPLES

Hereinafter, the present invention will be specifically explained based on Examples. Furthermore, the content of each component represents % by mass based on dry mass, and the water content represents mass parts relative to 100 mass parts by dry mass of the sheet unless otherwise stated in Tables. In addition, the contents of the component (a) and the component (b) in terms of the acid are also indicated respectively in Tables.

Furthermore, each of the measurements and the evaluations was carried out according to the methods and the standards described below.

<Evaluation of Tooth Whitening>

By measuring b* according to the method described below, whiteness of teeth (extracted teeth) before the above-mentioned treatment and after the above-mentioned treatment was evaluated.

Specifically, a digital camera D1x (manufactured by Nikon Corporation) and Ai AF Micro-Nikkor 105 mm F2.8D as a lens and Wireless Remote Speedlight SB-R200 (both manufactured by Nikon Corporation) as a stroboscopic light source were combined and used. The taken image was expressed with the L*a*b* color system using Adobe Photoshop (manufactured by Adobe Systems Incorporated), and the value of b* before attachment of the obtained sheet and b*10 minutes after the attachment were measured. Then, Δb* was calculated as a difference of b* between before attachment of the sheet and 10 minutes after the attachment {(b* after attachment)−(b* before attachment)}. A lower value of Δb*, i.e. a higher absolute value of Δb*, means whiter color.

<Evaluation of Tooth Gloss>

A method was used, in which surface reflected light intensity is measured by image analysis using polarized light. As a device for taking an evaluation image, a digital camera D2x (manufactured by Nikon Corporation) as a camera, Ai AF Micro-Nikkor 105 mm F2.8D as a lens and Wireless Remote Speedlight SB-R200 (both manufactured by Nikon Corporation) as a stroboscopic light source were combined and used. Plastic polarizing films (manufactured by Edmund Optics Inc.) were arranged in front of the light-emitting part of Speedlight and the lens so that the transmission axes would be crossed at a 30-degree angle, and photographs were taken. The average brightness of a highlight part was calculated from the photo image using Adobe Photoshop (manufactured by Adobe Systems Incorporated). A higher value of the brightness means an increase in gloss. The difference in brightness between before attachment of the obtained sheet (before the above-mentioned treatment) and 10 minutes after the attachment (after 14 times of the above-mentioned treatment) (brightness after attachment−brightness before attachment) was calculated as Δbrightness. A higher value of Δbrightness means an increase in gloss.

<Measurement of Water Content>

Measurement was carried out by a volume titration method using Karl Fischer Moisture Titrate (device for measurement of a minute amount of moisture, CA-200 type manufactured by Mitsubishi Chemical Analytech Co., Ltd.). Furthermore, the water content was measured for the sheet before attachment at 25° C. When the sheet was sealed in a bag including a resin sheet having an aluminum layer in the middle and stored before the attachment, the water content was measured for the sheet before being sealed in the bag.

<Measurement of Dissolution Time>

Each of the sheets of the layer (the layer (A) and the layer (B)) before lamination each having 10 mm×10 mm was immersed at 25° C. in water in so that the entire sheet was immersed, and the time until the sheet is dissolved and disappeared visually was assumed as the dissolution time. Furthermore, only in Example 1, water was applied at a speed of 0.1 g/min to the side of the layer (A), and the time until dissolution of the layer (B) (dissolution time AB) was measured, which was 11.5 minutes, the same as 11.5 minutes of the dissolution time shown in Table 1.

### Example 1

Each of the components was mixed properly using ethanol or purified water as a solvent and the base material solution (A) was produced so as to obtain the sheet (A) shown in Table 1. Then, the obtained base material solution (A) was coated on a PET film, and then dried to obtain the sheet (A). Similarly, each of the components was mixed properly using ethanol or purified water as a solvent and the base material solution (B) was produced so as to obtain the sheet (B) shown in Table 1. Then, the obtained base material solution (B) was coated on a PET film, and then dried, and detached from the PET film to obtain the sheet (B).

The obtained sheet (B) and sheet (A) were laminated, and pressed using a press roller, and detached from the PET film whereby to obtain the sheet of Example 1. Polyvalent cations were not blended in any of the base material solutions, and polyvalent cations such as magnesium, aluminum and calcium in the base material solution were measured by IPC emission spectrometry and were less than 0.02-fold mol relative to phytic acid.

Furthermore, 0.3 g water was dropped on the sheet inclined by 45 degrees from the horizontal surface with each layer upward, then the dropped water was rapidly collected, and the pH of each layer was measured by using a compact pH meter B-712 (HORIBA, Ltd.).

Then, the extracted teeth were soaked in deionized water for washing at room temperature (25° C.), and the side of the sheet (A) of the laminated sheet material was attached to the teeth so as to be in contact with the teeth, and stood for 10 minutes, and then immersed in the artificial saliva for about 3 hours. This operation was considered as a single treatment, and the treatment was carried out repeatedly 14 times.

Specifically, the procedures are as described below.

1. Extracted teeth were soaked in deionized water for washing at room temperature (25° C.), and attached with the sheet so as to contact the side of the exposed sheet (A) of the laminated sheet material with the teeth.

2. The side attached with the sheet material of the extracted teeth was brought into contact with 20 mm sponge supplying with artificial saliva at a speed of 0.1 g/min (communicative-porous sponge MAPS K001 manufactured by INOAC CORPORATION) for 10 minutes.

3. Then, the wreck of the remaining sheet was removed with light brushing and then was immersed in the artificial saliva for about 3 hours.

4. The treatments of 1 to 3 mentioned above were repeated 14 times.

Furthermore, the extracted teeth were human teeth that have not been subjected to stain removal treatment by abrasion and the like, and three extracted teeth per each test section were used. As the artificial saliva, an aqueous solution of calcium chloride (1.0 mM), potassium hydrogen phosphate (0.9 mM) and HEPES (4-(2-hydroxyethyl)-1-piperazine ethanesulfonic acid)(2.0 mM) adjusted to pH=7 with potassium hydroxide, was used.

Using the obtained sheet, the above-mentioned evaluations were carried out with respect to the teeth (extracted teeth) after the treatment. The results are shown in Table 1.

### Example 2

In a similar manner to that of Example 1, the base material solution (A) and the base material solution (B) were produced so as to obtain the sheet (A) and the sheet (B) shown in Table 1, and these sheets (A) and (B) were laminated and pressed. Then, the base material solution (C) was applied to one side of the sheet (B) opposite to the side of the laminated sheet (A), and the sheet (C) shown in Table 1 was laminated. The obtained sheet was dried to obtain the sheet of Example 2.

Using the obtained sheet, the above-mentioned evaluations were carried out with respect to the teeth (extracted teeth) subjected to similar treatment to those of Example 1. The results are shown in Table 1.

### Example 3

Procedures were carried out similarly to Example 2 except that a sheet (X) was further laminated on one side of the sheet (A) using the base material solution (X), and then the sheet (B) was further laminated to obtain the sheet of Example 3.

Using the obtained sheet, the above-mentioned evaluations were carried out with respect to the teeth (extracted teeth) subjected to similar treatment to those of Example 1. The results are shown in Table 1.

### Examples 4 to 6

The sheet (B) and the sheet (A) were laminated and pressed similarly to Example 1, to obtain each sheet shown in Table 1.

Using the obtained sheet, the above-mentioned evaluations were carried out with respect to the teeth (extracted teeth) subjected to similar treatment to those of Example 1. The results are shown in Table 1.

### Comparative Examples 1 to 3

According to Table 2, each base material solution was coated on a PET film, then dried, and detached from the PET film whereby to produce each sheet including single layer.

Using the obtained each sheet, the above-mentioned evaluations were carried out with respect to the teeth (extracted teeth) subjected to similar treatment to those of Example 1. The results are shown in Table 2.

TABLE 1

| | Example 1 | | Example 2 | | | Example 3 | | | | Example 4 | | Example 5 | | Example 6 | |
|---|---|---|---|---|---|---|---|---|---|---|---|---|---|---|---|
| Component | (A) | (B) | (A) | (B) | (C) | (A) | (X) | (B) | (C) | (A) | (B) | (A) | (B) | (A) | (B) |
| Anhydrous | 8 | — | 8 | — | — | 8 | — | — | — | 8 | — | 8 | — | 8 | — |
| Sodium | | | | | | | | | | | | | | | |
| pyro- | | | | | | | | | | | | | | | |
| phosphate | | | | | | | | | | | | | | | |
| Phytic acid | — | 15 | — | 15 | — | — | — | 15 | — | — | 15 | — | 15 | — | 15 |
| Sodium | — | q.s. | — | q.s. | — | — | — | q.s. | — | — | q.s. | — | q.s. | — | q.s. |
| hydroxide | | | | | | | | | | | | | | | |
| Hydrochloric | q.s. | — | q.s. | — | — | q.s. | — | — | — | q.s. | — | q.s. | — | q.s. | — |
| acid | | | | | | | | | | | | | | | |
| Conc. | 20 | 15 | 20 | 15 | — | 20 | 10 | 15 | — | 20 | 15 | 20 | 15 | 20 | 15 |
| glycerin | | | | | | | | | | | | | | | |
| Flavor | 1 | 1 | 1 | 1 | — | 1 | — | 1 | — | 1 | 1 | 1 | 1 | 1 | 1 |
| Poly- | 2.5 | 2.5 | 2.5 | 2.5 | — | 2.5 | — | 2.5 | — | 2.5 | 2.5 | 2.5 | 2.5 | 2.5 | 2.5 |
| glyceryl | | | | | | | | | | | | | | | |
| myristate | | | | | | | | | | | | | | | |
| Pullulan | Balance | Balance | Balance | Balance | — | Balance | 90 | Balance | — | Balance | Balance | Balance | Balance | Balance | Balance |
| Ethyl | — | — | — | — | 70 | — | — | — | 70 | — | — | — | — | — | — |
| cellulose | | | | | | | | | | | | | | | |
| Poly- | — | — | — | — | 30 | — | — | — | 30 | — | — | — | — | — | — |
| ethylene | | | | | | | | | | | | | | | |
| glycol | | | | | | | | | | | | | | | |
| Total | 100 | 100 | 100 | 100 | 100 | 100 | 100 | 100 | 100 | 100 | 100 | 100 | 100 | 100 | 100 |
| Amount in | 5.4 | 15 | 5.4 | 15 | — | 5.4 | — | 15 | — | 5.4 | 15 | 5.4 | 15 | 5.4 | 15 |
| terms of | | | | | | | | | | | | | | | |
| acid | | | | | | | | | | | | | | | |
| Thickness | 8 | 55 | 8 | 55 | 1 | 8 | 10 | 55 | 1 | 8 | 55 | 8 | 25 | 8 | 8 |
| (μm) | | | | | | | | | | | | | | | |
| Dissolu- | 1.5 | 10 | 1.5 | 10 | — | 1.5 | 1.8 | 10 | 1.8 | 1.5 | 10 | 1.5 | 4.5 | 1.5 | 1.5 |
| tion time | | | | | | | | | | | | | | | |
| (minute) | | | | | | | | | | | | | | | |
| pH | 9 | 4.5 | 9 | 4.5 | 7 | 9 | 7 | 4.5 | 7 | 7.5 | 5.5 | 9 | 4.5 | 9 | 4.5 |

| | | | | | | |
|---|---|---|---|---|---|---|
| Δb* | −1.55 | −2.47 | −2.53 | −1.32 | −1.28 | −0.34 |
| ΔBright- | 5.0 | 6.6 | 6.5 | 4.1 | 3.8 | 1.8 |
| ness | | | | | | |

TABLE 2

| | Comparative | Comparative | Comparative |
|---|---|---|---|
| Component | Example 1 | Example 2 | Example 3 |
| Anhydrous Sodium | 8 | — | 8 |
| pyrophosphate | | | |
| Phytic acid | — | 15 | 15 |
| Sodium hydroxide | — | q.s. | q.s. |
| Hydrochloric | q.s. | — | — |
| acid | | | |
| Conc. glycerin | 20 | 15 | 15 |
| Flavor | 1 | 1 | 1 |
| Polyglyceryl | 2.5 | 2.5 | 2.5 |
| myristate | | | |
| Pullulan | Balance | Balance | Balance |
| Total | 100 | 100 | 100 |
| Amount in terms | 5.4 | 15 | 20.4 |
| of acid | | | |
| Thickness (μm) | 63 | 63 | 63 |
| Dissolution time | 11 | 11 | 11 |
| (minute) | | | |
| pH | 9 | 4.5 | 6 |
| Δb* | −0.16 | −0.30 | −0.58 |
| ΔBrightness | 1.4 | 2.0 | 2.2 |

From the results of Tables 1 and 2, it is found that the adhesive sheet of the present invention exerts the excellent tooth whitening effect.

### Examples 7 to 45

According to Examples 1 to 3, each sheet shown in Tables 4, 9, 12, 14, 17 and 19 to 23 was obtained properly using the various base material solutions, and then each evaluation was properly carried out similarly to those described above. Any one of the sheets exerted the excellent tooth whitening effect and tooth gloss effect.

According to Examples 1 to 3, each sheet shown Tables 3, 5 to 8, 10 to 11, 13, 15 to 16 and 18 can be obtained properly using the various base material solutions. These sheets can also exert the excellent tooth whitening effect and tooth gloss effect.

TABLE 3

| | Example 7 | | Example 8 | | | Example 9 | | | |
|---|---|---|---|---|---|---|---|---|---|
| Component | (A) | (B) | (A) | (B) | (C) | (A) | (X) | (B) | (C) |
| Sodium tripolyphosphate | 8 | — | 8 | — | — | 8 | — | — | — |
| Phytic acid | — | 15 | — | 15 | — | — | — | 15 | — |
| Sodium hydroxide | — | q.s. | — | q.s. | — | — | — | q.s. | — |
| Hydrochloric acid | q.s. | — | q.s. | — | — | q.s. | — | — | — |
| Conc. glycerin | 20 | 15 | 20 | 15 | — | 20 | 10 | 15 | — |
| Flavor | 1 | 1 | 1 | 1 | — | 1 | — | 1 | — |
| Polyglyceryl myristate | 2.5 | 2.5 | 2.5 | 2.5 | — | 2.5 | — | 2.5 | — |
| Pullulan | Balance | Balance | Balance | Balance | — | Balance | 90 | Balance | — |
| Ethyl cellulose | — | — | — | — | 70 | — | — | — | 70 |
| Polyethylene glycol | — | — | — | — | 30 | — | — | — | 30 |
| Total | 100 | 100 | 100 | 100 | 100 | 100 | 100 | 100 | 100 |
| Amount in terms of acid | 5.6 | 15 | 5.6 | 15 | — | 5.6 | — | 15 | — |
| Thickness (μm) | 8 | 55 | 8 | 55 | 1 | 8 | 10 | 55 | 1 |
| pH | 9 | 4.5 | 9 | 4.5 | 7 | 9 | 7 | 4.5 | 7 |

TABLE 4

Example 10

| Component | (A) | (B) |
|---|---|---|
| Sodium lauryl | 8 | — |
| sulfate | | |
| Phytic acid | — | 15 |
| Sodium hydroxide | — | q.s. |
| Hydrochloric acid | q.s. | — |
| Conc. glycerin | 20 | 15 |
| Flavor | 1 | 1 |
| Polyglyceryl | 2.5 | 2.5 |
| myristate | | |
| Pullulan | Balance | Balance |
| Ethyl cellulose | — | — |
| Polyethylene glycol | — | — |
| Total | 100 | 100 |
| Amount in terms of | 7.4 | 15 |
| acid | | |
| Thickness (μm) | 8 | 55 |
| Dissolution time | 1.5 | 10 |
| (minute) | | |
| pH | 9 | 4.5 |

| | |
|---|---|
| Δb* | −1.41 |
| ΔBrightness | 4.5 |

TABLE 5

| | Example 11 | | | Example 12 | | | |
|---|---|---|---|---|---|---|---|
| Component | (A) | (B) | (C) | (A) | (X) | (B) | (C) |
| Sodium lauryl | 8 | — | — | 8 | — | — | — |
| sulfate | | | | | | | |
| Phytic acid | — | 15 | — | — | — | 15 | — |
| Sodium | — | q.s | — | — | — | q.s | — |
| hydroxide | | | | | | | |
| Hydrochloric | q.s. | — | — | q.s | — | — | — |
| acid | | | | | | | |
| Conc. glycerin | 20 | 15 | — | 20 | 10 | 15 | — |
| Flavor | 1 | 1 | — | 1 | — | 1 | — |
| Polyglyceryl | 2.5 | 2.5 | — | 2.5 | — | 2.5 | — |
| myristate | | | | | | | |
| Pullulan | Bal- | Balance | — | Balance | 90 | Balance | — |
| | ance | | | | | | |
| Ethyl cellulose | — | — | 70 | — | — | — | 70 |
| Polyethylene | — | — | 30 | — | — | — | 30 |
| glycol | | | | | | | |
| Total | 100 | 100 | 100 | 100 | 100 | 100 | 100 |
| Amount in | 7.4 | 15 | — | 7.4 | — | 15 | — |
| terms of acid | | | | | | | |
| Thickness (μm) | 8 | 55 | 1 | 8 | 10 | 55 | 1 |
| pH | 9 | 4.5 | 7 | 9 | 7 | 4.5 | 7 |

TABLE 6

| | Example 13 | | Example 14 | | | Example 15 | | | |
|---|---|---|---|---|---|---|---|---|---|
| Component | (A) | (B) | (A) | (B) | (C) | (A) | (X) | (B) | (C) |
| Sodium | 8 | — | 8 | — | — | 8 | — | — | — |
| pyrophosphate | | | | | | | | | |
| Malic acid | — | 15 | — | 15 | — | — | — | 15 | — |
| Sodium hydroxide | — | q.s. | — | q.s. | — | — | — | q.s. | — |
| Hydrochloric acid | q.s. | — | q.s. | — | — | q.s. | — | — | — |
| Conc. glycerin | 20 | 15 | 20 | 15 | — | 20 | 10 | 15 | — |
| Flavor | 1 | 1 | 1 | 1 | — | 1 | — | 1 | — |
| Polyglyceryl | 2.5 | 2.5 | 2.5 | 2.5 | — | 2.5 | — | 2.5 | — |
| myristate | | | | | | | | | |
| Pullulan | Balance | Balance | Balance | Balance | — | Balance | 90 | Balance | — |
| Ethyl cellulose | — | — | — | — | 70 | — | — | — | 70 |
| Polyethylene glycol | — | — | — | — | 30 | — | — | — | 30 |
| Total | 100 | 100 | 100 | 100 | 100 | 100 | 100 | 100 | 100 |
| Amount in terms of | 5.4 | 15 | 5.4 | 15 | — | 5.4 | — | 15 | — |
| acid | | | | | | | | | |
| Thickness (μm) | 8 | 55 | 8 | 55 | 1 | 8 | 10 | 55 | 1 |
| pH | 9 | 4.5 | 9 | 4.5 | 7 | 9 | 7 | 4.5 | 7 |

TABLE 7

| | Example 16 | | Example 17 | | | Example 18 | | | |
|---|---|---|---|---|---|---|---|---|---|
| Component | (A) | (B) | (A) | (B) | (C) | (A) | (X) | (B) | (C) |
| Sodium | 8 | — | 8 | — | — | 8 | — | — | — |
| tripolyphosphate | | | | | | | | | |
| Malic acid | — | 15 | — | 15 | — | — | — | 15 | — |
| Sodium hydroxide | — | q.s. | — | q.s. | — | — | — | q.s. | — |
| Hydrochloric acid | q.s. | — | q.s. | — | — | q.s. | — | — | — |
| Conc. glycerin | 20 | 15 | 20 | 15 | — | 20 | 10 | 15 | — |
| Flavor | 1 | 1 | 1 | 1 | — | 1 | — | 1 | — |
| Polyglyceryl | 2.5 | 2.5 | 2.5 | 2.5 | — | 2.5 | — | 2.5 | — |
| myristate | | | | | | | | | |
| Pullulan | Balance | Balance | Balance | Balance | — | Balance | 90 | Balance | — |
| Ethyl cellulose | — | — | — | — | 70 | — | — | — | 70 |
| Polyethylene glycol | — | — | — | — | 30 | — | — | — | 30 |
| Total | 100 | 100 | 100 | 100 | 100 | 100 | 100 | 100 | 100 |
| Amount in terms of | 5.6 | 15 | 5.6 | 15 | — | 5.6 | — | 15 | — |
| acid | | | | | | | | | |
| Thickness (μm) | 8 | 55 | 8 | 55 | 1 | 8 | 10 | 55 | 1 |
| pH | 9 | 4.5 | 9 | 4.5 | 7 | 9 | 7 | 4.5 | 7 |

TABLE 8

| | Example 19 | | Example 20 | | | Example 21 | | | |
|---|---|---|---|---|---|---|---|---|---|
| Component | (A) | (B) | (A) | (B) | (C) | (A) | (X) | (B) | (C) |
| Sodium lauryl sulfate | 8 | — | 8 | — | — | 8 | — | — | — |
| Malic acid | — | 15 | — | 15 | — | — | — | 15 | — |
| Sodium hydroxide | — | q.s. | — | q.s. | — | — | — | q.s. | — |
| Hydrochloric acid | q.s. | — | q.s. | — | — | q.s. | — | — | — |
| Conc. glycerin | 20 | 15 | 20 | 15 | — | 20 | 10 | 15 | — |
| Flavor | 1 | 1 | 1 | 1 | — | 1 | — | 1 | — |
| Polyglyceryl myristate | 2.5 | 2.5 | 2.5 | 2.5 | — | 2.5 | — | 2.5 | — |
| Pullulan | Balance | Balance | Balance | Balance | — | Balance | 90 | Balance | — |
| Ethyl cellulose | — | — | — | — | 70 | — | — | — | 70 |
| Polyethylene glycol | — | — | — | — | 30 | — | — | — | 30 |
| Total | 100 | 100 | 100 | 100 | 100 | 100 | 100 | 100 | 100 |
| Amount in terms of acid | 7.4 | 15 | 7.4 | 15 | — | 7.4 | — | 15 | — |
| Thickness (μm) | 8 | 55 | 8 | 55 | 1 | 8 | 10 | 55 | 1 |
| pH | 9 | 4.5 | 9 | 4.5 | 7 | 9 | 7 | 4.5 | 7 |

TABLE 9

Example 22

| Component | (A) | (B) |
|---|---|---|
| Sodium | 8 | — |
| pyrophosphate | | |
| Hexametaphosphoric | — | 15 |
| acid | | |
| Sodium hydroxide | — | q.s. |
| Hydrochloric acid | q.s. | — |
| Conc. glycerin | 20 | 15 |
| Flavor | 1 | 1 |
| Polyglyceryl | 2.5 | 2.5 |
| myristate | | |
| Pullulan | Balance | Balance |
| Ethyl cellulose | — | — |
| Polyethylene glycol | — | — |
| Total | 100 | 100 |
| Amount in terms of | 5.4 | 15 |
| acid | | |
| Thickness (μm) | 8 | 55 |
| Dissolution time | 1.5 | 10 |
| (minute) | | |
| pH | 9 | 4.5 |

| | |
|---|---|
| Δb* | −1.35 |
| ΔBrightness | 4.4 |

TABLE 10

| | Example 23 | | | Example 24 | | | |
|---|---|---|---|---|---|---|---|
| Component | (A) | (B) | (C) | (A) | (X) | (B) | (C) |
| Sodium | 8 | — | — | 8 | — | — | — |
| pyro- | | | | | | | |
| phosphate | | | | | | | |
| Hexameta- | — | 15 | — | — | — | 15 | — |
| phosphoric | | | | | | | |
| acid | | | | | | | |
| Sodium | — | q.s. | — | — | — | q.s. | — |
| hydroxide | | | | | | | |
| Hydrochloric | q.s. | — | — | q.s. | — | — | — |
| acid | | | | | | | |
| Conc. | 20 | 15 | — | 20 | 10 | 15 | — |
| glycerin | | | | | | | |
| Flavor | 1 | 1 | — | 1 | — | 1 | — |
| Polyglyceryl | 2.5 | 2.5 | — | 2.5 | — | 2.5 | — |
| myristate | | | | | | | |
| Pullulan | Balance | Balance | — | Balance | 90 | Balance | — |
| Ethyl | — | — | 70 | — | — | — | 70 |
| cellulose | | | | | | | |
| Polyethylene | — | — | 30 | — | — | — | 30 |
| glycol | | | | | | | |
| Total | 100 | 100 | 100 | 100 | 100 | 100 | 100 |
| Amount in | 5.4 | 15 | — | 5.4 | — | 15 | — |
| terms of | | | | | | | |
| acid | | | | | | | |
| Thickness | 8 | 55 | 1 | 8 | 10 | 55 | 1 |
| (μm) | | | | | | | |
| pH | 9 | 4.5 | 7 | 9 | 7 | 4.5 | 7 |

TABLE 11

| | Example 25 | | Example 26 | | | Example 27 | | | |
|---|---|---|---|---|---|---|---|---|---|
| Component | (A) | (B) | (A) | (B) | (C) | (A) | (X) | (B) | (C) |
| Sodium | 8 | — | 8 | — | — | 8 | — | — | — |
| tripolyphosphate | | | | | | | | | |
| Hexametaphosphoric | — | 15 | — | 15 | — | — | — | 15 | — |
| acid | | | | | | | | | |
| Sodium hydroxide | — | q.s. | — | q.s. | — | — | — | q.s. | — |
| Hydrochloric acid | q.s. | — | q.s. | — | — | q.s. | — | — | — |
| Conc. glycerin | 20 | 15 | 20 | 15 | — | 20 | 9 | 15 | — |
| Flavor | 1 | 1 | 1 | 1 | — | 1 | — | 1 | — |
| Polyglyceryl | 2.5 | 2.5 | 2.5 | 2.5 | — | 2.5 | — | 2.5 | — |
| myristate | | | | | | | | | |
| Pullulan | Balance | Balance | Balance | Balance | — | Balance | 81 | Balance | — |
| Ethyl cellulose | — | — | — | — | 70 | — | — | — | 70 |
| Polyethylene glycol | — | — | — | — | 30 | — | — | — | 30 |
| Total | 100 | 100 | 100 | 100 | 100 | 100 | 100 | 100 | 100 |
| Amount in terms of | 5.6 | 15 | 5.6 | 15 | — | 5.6 | — | 15 | — |
| acid | | | | | | | | | |
| pH | 9 | 4.5 | 9 | 4.5 | 7 | 9 | 7 | 4.5 | 7 |
| Water content | 14 | 14 | 14 | 14 | 14 | 14 | 14 | 14 | 14 |
| relative to 100 mass | | | | | | | | | |
| parts by dry mass of | | | | | | | | | |
| sheet (mass parts) | | | | | | | | | |

TABLE 12

Example 28

| Component | (A) | (B) |
|---|---|---|
| Sodium lauryl | 8 | — |
| sulfate | | |
| Hexametaphosphoric | — | 15 |
| acid | | |
| Sodium hydroxide | — | q.s. |
| Hydrochloric acid | q.s. | — |
| Conc. glycerin | 20 | 15 |
| Flavor | 1 | 1 |
| Polyglyceryl | 2.5 | 2.5 |
| myristate | | |
| Pullulan | Balance | Balance |
| Ethyl cellulose | — | — |
| Polyethylene glycol | — | — |
| Total | 100 | 100 |
| Amount in terms of | 7.4 | 15 |
| acid | | |
| Thickness (μm) | 8 | 55 |
| Dissolution time | 1.5 | 10 |
| (minute) | | |
| pH | 9 | 4.5 |

| | |
|---|---|
| Δb* | −1.11 |
| ΔBrightness | 3.4 |

TABLE 13

| | Example 29 | | | Example 30 | | | |
|---|---|---|---|---|---|---|---|
| Component | (A) | (B) | (C) | (A) | (X) | (B) | (C) |
| Sodium lauryl sulfate | 8 | — | — | 8 | — | — | — |
| Hexametaphosphoric acid | — | 15 | — | — | — | 15 | — |
| Sodium hydroxide | — | q.s. | — | — | — | q.s. | — |
| Hydrochloric acid | q.s. | — | — | q.s. | — | — | — |
| Conc. glycerin | 20 | 15 | — | 20 | 10 | 15 | — |
| Flavor | 1 | 1 | — | 1 | — | 1 | — |
| Polyglyceryl myristate | 2.5 | 2.5 | — | 2.5 | — | 2.5 | — |
| Pullulan | Balance | Balance | — | Balance | 90 | Balance | — |
| Ethyl cellulose | — | — | 70 | — | — | — | 70 |
| Polyethylene glycol | — | — | 30 | — | — | — | 30 |
| Total | 100 | 100 | 100 | 100 | 100 | 100 | 100 |
| Amount in terms of acid | 7.4 | 15 | — | 7.4 | — | 15 | — |
| Thickness (μm) | 8 | 55 | 1 | 8 | 10 | 55 | 1 |
| pH | 9 | 4.5 | 7 | 9 | 7 | 4.5 | 7 |

TABLE 14

Example 31

| Component | (A) | (B) |
|---|---|---|
| Sodium | 8 | — |
| pyrophosphate | | |
| Citric acid | — | 15 |
| Sodium hydroxide | — | q.s. |
| Hydrochloric acid | q.s. | — |
| Conc. glycerin | 20 | 15 |
| Flavor | 1 | 1 |
| Polyglyceryl | 2.5 | 2.5 |
| myristate | | |
| Pullulan | Balance | Balance |
| Ethyl cellulose | — | — |
| Polyethylene glycol | — | — |
| Total | 100 | 100 |
| Amount in terms of | 5.4 | 15 |
| acid | | |
| Thickness (μm) | 8 | 55 |
| Dissolution time | 1.5 | 10 |
| (minute) | | |
| pH | 9 | 4.5 |

| | |
|---|---|
| Δb* | −0.83 |
| ΔBrightness | 2.7 |

TABLE 15

| | Example 32 | | | Example 33 | | | |
|---|---|---|---|---|---|---|---|
| Component | (A) | (B) | (C) | (A) | (X) | (B) | (C) |
| Sodium | 8 | — | — | 8 | — | — | — |
| pyro- | | | | | | | |
| phosphate | | | | | | | |
| Citric acid | — | 15 | — | — | — | 15 | — |
| Sodium | — | q.s. | — | — | — | q.s. | — |
| hydroxide | | | | | | | |
| Hydrochloric | q.s. | — | — | q.s. | — | — | — |
| acid | | | | | | | |
| Conc. | 20 | 15 | — | 20 | 10 | 15 | — |
| glycerin | | | | | | | |
| Flavor | 1 | 1 | — | 1 | — | 1 | — |
| Polyglyceryl | 2.5 | 2.5 | — | 2.5 | — | 2.5 | — |
| myristate | | | | | | | |
| Pullulan | Balance | Balance | — | Balance | 90 | Balance | — |
| Ethyl | — | — | 70 | — | — | — | 70 |
| cellulose | | | | | | | |
| Polyethylene | — | — | 30 | — | — | — | 30 |
| glycol | | | | | | | |
| Total | 100 | 100 | 100 | 100 | 100 | 100 | 100 |
| Amount in | 5.4 | 15 | — | 5.4 | — | 15 | — |
| terms of acid | | | | | | | |
| Thickness | 8 | 55 | 1 | 8 | 10 | 55 | 1 |
| (μm) | | | | | | | |
| pH | 9 | 4.5 | 7 | 9 | 7 | 4.5 | 7 |

TABLE 16

| | | |
|---|---|---|
| Example 34 | Example 35 | Example 36 |

| Component | (A) | (B) | (A) | (B) | (C) | (A) | (X) | (B) | (C) |
|---|---|---|---|---|---|---|---|---|---|
| Sodium | 8 | — | 8 | — | — | 8 | — | — | — |
| tripolyphosphate | | | | | | | | | |
| Citric acid | — | 15 | — | 15 | — | — | — | 15 | — |
| Sodium hydroxide | — | q.s. | — | q.s. | — | — | — | q.s. | — |
| Hydrochloric acid | q.s. | — | q.s. | — | — | q.s. | — | — | — |
| Conc. glycerin | 20 | 15 | 20 | 15 | — | 20 | 10 | 15 | — |
| Flavor | 1 | 1 | 1 | 1 | — | 1 | — | 1 | — |
| Polyglyceryl | 2.5 | 2.5 | 2.5 | 2.5 | — | 2.5 | — | 2.5 | — |
| myristate | | | | | | | | | |
| Pullulan | Balance | Balance | Balance | Balance | — | Balance | 90 | Balance | — |
| Ethyl cellulose | — | — | — | — | 70 | — | — | — | 70 |
| Polyethylene glycol | — | — | — | — | 30 | — | — | — | 30 |
| Total | 100 | 100 | 100 | 100 | 100 | 100 | 100 | 100 | 100 |
| Amount in terms of | 5.6 | 15 | 5.6 | 15 | — | 5.6 | — | 15 | — |
| acid | | | | | | | | | |
| Thickness (μm) | 8 | 55 | 8 | 55 | 1 | 8 | 10 | 55 | 1 |
| pH | 9 | 4.5 | 9 | 4.5 | 7 | 9 | 7 | 4.5 | 7 |

TABLE 17

Example 37

| Component | (A) | (B) |
|---|---|---|
| Sodium lauryl | 8 | — |
| sulfate | | |
| Citric acid | — | 15 |
| Sodium hydroxide | — | q.s. |
| Hydrochloric acid | q.s. | — |
| Conc. glycerin | 20 | 15 |
| Flavor | 1 | 1 |
| Polyglyceryl | 2.5 | 2.5 |
| myristate | | |
| Pullulan | Balance | Balance |
| Ethyl cellulose | — | — |
| Polyethylene glycol | — | — |
| Total | 100 | 100 |
| Amount in terms of | 7.4 | 15 |
| acid | | |
| Thickness (μm) | 8 | 55 |
| Dissolution time | 1.5 | 10 |
| (minute) | | |
| pH | 9 | 4.5 |

| | |
|---|---|
| Δb* | −0.68 |
| ΔBrightness | 2.3 |

TABLE 18

| | Example 38 | | | Example 39 | | | |
|---|---|---|---|---|---|---|---|
| Component | (A) | (B) | (C) | (A) | (X) | (B) | (C) |
| Sodium | 8 | — | — | 8 | — | — | — |
| lauryl | | | | | | | |
| sulfate | | | | | | | |
| Citric acid | — | 15 | — | — | — | 15 | — |
| Sodium | — | q.s. | — | — | — | q.s. | — |
| hydroxide | | | | | | | |
| Hydrochloric | q.s. | — | — | q.s. | — | — | — |
| acid | | | | | | | |
| Conc. | 20 | 15 | — | 20 | 10 | 15 | — |
| glycerin | | | | | | | |
| Flavor | 1 | 1 | — | 1 | — | 1 | — |
| Polyglyceryl | 2.5 | 2.5 | — | 2.5 | — | 2.5 | — |
| myristate | | | | | | | |
| Pullulan | Balance | Balance | — | Balance | 90 | Balance | — |
| Ethyl | — | — | 70 | — | — | — | 70 |
| cellulose | | | | | | | |
| Polyethylene | — | — | 30 | — | — | — | 30 |
| glycol | | | | | | | |
| Total | 100 | 100 | 100 | 100 | 100 | 100 | 100 |
| Amount in | 7.36 | 15 | — | 7.36 | 13 | 15 | — |
| terms of acid | | | | | | | |
| Thickness | 8 | 55 | 1 | 8 | 10 | 55 | 1 |
| (μm) | | | | | | | |
| Water | 14 | 14 | 14 | 14 | 14 | 14 | 14 |
| content | | | | | | | |
| relative to | | | | | | | |
| 100 mass | | | | | | | |
| parts by | | | | | | | |
| dry mass | | | | | | | |
| of sheet | | | | | | | |
| (mass parts) | | | | | | | |
| pH | 9 | 4.5 | 7 | 9 | 7 | 4.5 | 7 |

TABLE 19

| | Example 1 | | Example 4 | | Example 40 | |
|---|---|---|---|---|---|---|
| Component | (A) | (B) | (A) | (B) | (A) | (B) |
| Anhydrous | 8 | — | 8 | — | 8 | — |
| Sodium | | | | | | |
| pyrophosphate | | | | | | |
| Phytic acid | — | 15 | — | 15 | — | 15 |
| Sodium | — | q.s. | — | q.s. | — | q.s. |
| hydroxide | | | | | | |
| Hydrochloric | q.s. | — | q.s. | — | q.s. | — |
| acid | | | | | | |
| Conc. glycerin | 20 | 15 | 20 | 15 | 20 | 15 |
| Flavor | 1 | 1 | 1 | 1 | 1 | 1 |
| Polyglyceryl | 2.5 | 2.5 | 2.5 | 2.5 | 2.5 | 2.5 |
| myristate | | | | | | |
| Pullulan | Balance | Balance | Balance | Balance | Balance | Balance |
| Ethyl cellulose | — | — | — | — | — | — |
| Polyethylene | — | — | — | — | — | — |
| glycol | | | | | | |
| Total | 100 | 100 | 100 | 100 | 100 | 100 |
| Amount in | 5.4 | 15 | 5.4 | 15 | 5.4 | 15 |
| terms of acid | | | | | | |
| Thickness (μm) | 8 | 55 | 8 | 55 | 8 | 55 |
| Dissolution | 1.5 | 10 | 1.5 | 10 | 1.5 | 10 |
| time (minute) | | | | | | |
| pH | 9 | 4.5 | 7.5 | 5.5 | 9.5 | 6 |

| | | | |
|---|---|---|---|
| Δb* | −1.55 | −1.32 | −0.87 |
| ΔBrightness | 5.0 | 4.1 | 1.0 |

TABLE 20

| | Example 2 | | | Example 41 | | |
|---|---|---|---|---|---|---|
| Component | (A) | (B) | (C) | (A) | (B) | (C) |
| Anhydrous Sodium | 8 | — | — | 8 | — | — |
| pyrophosphate | | | | | | |
| Phytic acid | — | 15 | — | — | 15 | — |
| Sodium hydroxide | — | q.s. | — | — | q.s. | — |
| Hydrochloric acid | q.s. | — | — | q.s. | — | — |
| Conc. glycerin | 20 | 15 | — | 20 | 15 | — |
| Flavor | 1 | 1 | — | 1 | 1 | — |
| Polyglyceryl | 2.5 | 2.5 | — | 2.5 | 2.5 | — |
| myristate | | | | | | |
| Pullulan | Balance | Balance | — | — | — | — |
| Hydroxyethyl | — | — | | — | — | |
| cellulose | | | | | | |
| Hydroxypropyl | — | — | | — | — | |
| cellulose | | | | | | |
| Hydroxy- | — | — | | Balance | Balance | |
| propylmethyl | | | | | | |
| cellulose | | | | | | |
| Ethyl cellulose | — | — | 70 | — | — | 70 |
| Polyethylene glycol | — | — | 30 | — | — | 30 |
| Total | 100 | 100 | 100 | 100 | 100 | 100 |
| Amount in terms of | 5.4 | 15 | — | 5.4 | 15 | — |
| acid | | | | | | |
| Thickness (μm) | 8 | 55 | 1 | 8 | 55 | 1 |
| Dissolution time | 1.5 | 10 | — | 5 | 30 | — |
| (minute) | | | | | | |
| pH | 9 | 4.5 | 7 | 9 | 4.5 | 7 |

| | | |
|---|---|---|
| Δb* | −2.47 | −3.05 |
| ΔBrightness | 6.6 | 2.0 |

TABLE 21

| | Example 1 | | Example 5 | | Example 6 | | Example 42 | |
|---|---|---|---|---|---|---|---|---|
| Component | (A) | (B) | (A) | (B) | (A) | (B) | (A) | (B) |
| Anhydrous Sodium | 8 | — | 8 | — | 8 | — | 8 | — |
| pyrophosphate | | | | | | | | |
| Phytic acid | — | 15 | — | 15 | — | 15 | — | 15 |
| Sodium hydroxide | — | q.s. | — | q.s. | — | q.s. | — | q.s. |
| Hydrochloric acid | q.s. | — | q.s. | — | q.s. | — | q.s. | — |
| Conc. glycerin | 20 | 15 | 20 | 15 | 20 | 15 | 20 | 15 |
| Flavor | 1 | 1 | 1 | 1 | 1 | 1 | 1 | 1 |
| Polyglyceryl | 2.5 | 2.5 | 2.5 | 2.5 | 2.5 | 2.5 | 2.5 | 2.5 |
| myristate | | | | | | | | |
| Pullulan | Balance | Balance | Balance | Balance | Balance | Balance | Balance | Balance |
| Ethyl cellulose | — | — | — | — | — | — | — | — |
| Polyethylene glycol | — | — | — | — | — | — | — | — |
| Total | 100 | 100 | 100 | 100 | 100 | 100 | 100 | 100 |
| Amount in terms of | 5.4 | 15 | 5.4 | 15 | 5.4 | 15 | 5.4 | 15 |
| acid | | | | | | | | |
| Thickness (μm) | 8 | 55 | 8 | 25 | 8 | 8 | 15 | 55 |
| Dissolution time | 1.5 | 10 | 1.5 | 4.5 | 1.5 | 1.5 | 3 | 10 |
| (minute) | | | | | | | | |
| pH | 9 | 4.5 | 9 | 4.5 | 9 | 4.5 | 9 | 4.5 |

| | | | | |
|---|---|---|---|---|
| Δb* | −1.55 | −1.28 | −0.34 | −2.81 |
| ΔBrightness | 5.0 | 3.8 | 1.8 | 4.1 |

TABLE 22

| | Example 2 | | | Example 43 | | |
|---|---|---|---|---|---|---|
| Component | (A) | (B) | (C) | (A) | (B) | (C) |
| Anhydrous Sodium | 8 | — | — | 12 | — | — |
| pyrophosphate | | | | | | |
| Phytic acid | — | 15 | — | — | 26 | — |
| Sodium hydroxide | — | q.s. | — | — | q.s. | — |
| Hydrochloric | q.s. | — | — | q.s. | — | — |
| acid | | | | | | |
| Conc. glycerin | 20 | 15 | — | 20 | 15 | — |
| Flavor | 1 | 1 | — | 1 | 1 | — |
| Polyglyceryl | 2.5 | 2.5 | — | 2.5 | 2.5 | — |
| myristate | | | | | | |
| Pullulan | Balance | Balance | — | Balance | Balance | — |
| Ethyl cellulose | — | — | 70 | — | — | 70 |
| Shellac | — | — | — | — | — | — |
| Polyethylene | — | — | 30 | — | — | 30 |
| glycol | | | | | | |
| Total | 100 | 100 | 100 | 100 | 100 | 100 |
| Amount in terms | 5.4 | 15 | — | 8.0 | 26 | — |
| of acid | | | | | | |
| Thickness (μm) | 8 | 55 | 1 | 8 | 55 | 1 |
| Dissolution time | 1.5 | 10 | — | 1.5 | 10 | — |
| (minute) | | | | | | |
| pH | 9 | 4.5 | 7 | 9 | 4.5 | 7 |

| | | |
|---|---|---|
| Δb* | −2.47 | −2.16 |
| ΔBrightness | 6.6 | 1.5 |

TABLE 23

| | Example 2 | | | Example 44 | | | | Example 45 | | | |
|---|---|---|---|---|---|---|---|---|---|---|---|
| Component | (A) | (B) | (C) | (A) | (X) | (B) | (C) | (A) | (X) | (B) | (C) |
| Anhydrous | 8 | — | — | 8 | — | — | — | 8 | — | — | — |
| Sodium | | | | | | | | | | | |
| pyrophosphate | | | | | | | | | | | |
| Phytic acid | — | 15 | — | — | — | 15 | — | — | — | 15 | — |
| Sodium | — | q.s. | — | — | — | q.s. | — | — | — | q.s. | — |
| hydroxide | | | | | | | | | | | |
| Hydrochloric | q.s. | — | — | q.s. | — | — | — | q.s. | — | — | — |
| acid | | | | | | | | | | | |
| Conc. glycerin | 20 | 15 | — | 20 | — | 15 | — | 20 | — | 15 | — |
| Flavor | 1 | 1 | — | 1 | — | 1 | — | 1 | — | 1 | — |
| Polyglyceryl | 2.5 | 2.5 | — | 2.5 | — | 2.5 | — | 2.5 | — | 2.5 | — |
| myristate | | | | | | | | | | | |
| Pullulan | Balance | Balance | — | Balance | — | Balance | — | Balance | — | Balance | — |
| Ethyl cellulose | — | — | 70 | — | 70 | — | 70 | — | — | — | 70 |
| Shellac | — | — | — | — | — | — | — | — | 100 | — | — |
| Polyethylene | — | — | 30 | — | 30 | — | 30 | — | — | — | 30 |
| glycol | | | | | | | | | | | |
| Total | 100 | 100 | 100 | 100 | 100 | 100 | 100 | 100 | 100 | 100 | 100 |
| Amount in terms | 5.4 | 15 | — | 5.4 | — | 15 | — | 5.4 | — | 15 | — |
| of acid | | | | | | | | | | | |
| Thickness (μm) | 8 | 55 | 1 | 8 | 1.5 | 55 | 1 | 8 | 1.5 | 55 | 1 |
| Dissolution | 1.5 | 10 | — | 1.5 | — | 10 | — | 1.5 | — | 10 | — |
| time (minute) | | | | | | | | | | | |
| pH | 9 | 4.5 | 7 | 9 | 7 | 4.5 | 7 | 9 | 7 | 4.5 | 7 |

| | | | |
|---|---|---|---|
| Δb* | −2.47 | −2.5 | −2.5 |
| ΔBrightness | 6.6 | 6.6 | 6.6 |

### Example 46

Each of the components was mixed and the base material solution (A) was produced properly using ethanol or purified water as a solvent so as to obtain the sheet (A) shown in Table 24. Then, the obtained base material solution (A) was coated on a PET film, and then dried to achieve the water content shown in Table 25 to obtain the sheet (A). Similarly, each of the components was mixed the base material solution (B) was produced properly using ethanol or purified water as a solvent so as to obtain the sheet (B) shown in Table 24. Then, the obtained base material solution (B) was coated on a PET film, and then dried to achieve the water content shown in Table 25, and detached from the PET film to obtain the sheet (B).

The obtained sheets (B) and (A) were laminated, and pressed using a press roller, and detached from the PET film, and then a base material solution (C) shown in Table 24 was coated on one side of the sheet (A) that was the opposite side to the side laminated on sheet (B), and dried so that the water content in the entire laminated sheet became the amount shown in Table 25, whereby to obtain the laminated sheet of Example 46. Polyvalent cations were not blended in any of the base material solutions, and polyvalent cations such as magnesium, aluminum and calcium in the base material solution were measured by IPC emission spectrometry and were less than 0.02-fold mol relative to phytic acid.

Furthermore, 0.3 g water was dropped on the sheet inclined by 45 degrees from the horizontal surface with each layer upward, and then the dropped water was rapidly collected, and the pH of each layer was measured by using a compact pH meter B-712 (HORIBA, Ltd.).

The obtained laminated sheet was sealed in a pillow bag including a resin sheet of multi-layer structure in which the innermost layer was polyethylene (polyethylene terephthalate 12 μm/polyethylene chloride resin 15 μm/aluminum 7 μm/polyethylene chloride resin 20 μm/low density polyethylene 60 μm) while being supported with a PET film, and stored at 25° C. for 1 week.

The pH of the sheet after storage was measured by the method described below, and time-dependent pH change was observed with the measurement method 1 shown below.

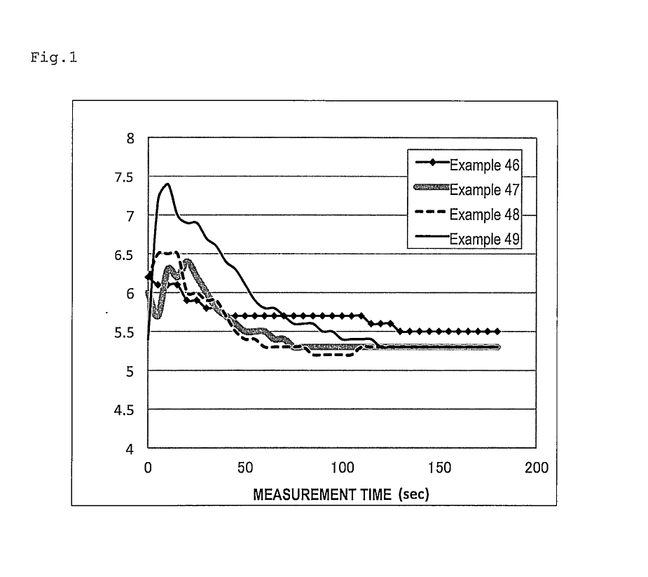

The results are illustrated in FIG. 1. Furthermore, the water contents shown in Table 25 are amounts of water before sealed in the pillow bag.

<Measurement Method 1 for Time-Dependent pH Change>

The sheet removed from the pillow bag was detached from the PET film, and then pH was measured using a pH meter (AS-212, HORIBA, Ltd.). Specifically, 0.1 g deionized water was dropped on the sensor (electrode section) of the pH meter at 25° C., further 10 mm×10 mm of the sheet was placed so that the layer (A) was located at the side of deionized water, and the value of pH shown in the pH meter was measured every 5 seconds wherein the time when the sheet was placed was taken as the initiation time.

<Measurement Method 2 for Time-Dependent pH Change>

The sheet removed from the pillow bag was detached from the PET film and then attached to teeth in the oral cavity in which the electrode of a pH meter (PH-201Z portable pH meter, Chemical Instruments Co., LTD.) was fixed, and the pH was measured. Specifically, a stent made of silicone covering two teeth of the lateral incisor of the right lower jaw and the canine tooth of the right lower jaw arranged in line of a test subject with a thin film, was prepared, the electrode (custom-made pH antimony electrode SP-Sb-010, Chemical Instruments Co., LTD.) of the pH meter was fixed between the two teeth mounted with the silicone stent such that the electrode came out on the side of the surface of the stent made of silicone, and the sheet was attached on the surface of the silicone. The pH was measured every 100 milliseconds from the time immediately after the attachment.

### Examples 47 to 49

According to similar procedures to those of Example 46, the base material solutions (A) to (C) shown in Table 24 were produced, and dried to achieve the water contents shown in Table 25, whereby to obtain each laminated sheet. Furthermore, Examples 47 to 49 were produced while preventing an increase of the water content by being sealed in a pillow bag before absorbing moisture after the drying.

Using the obtained laminated sheet, the pH change was observed with the measurement method 1 similarly to Example 46. The results are illustrated in FIG. 1.

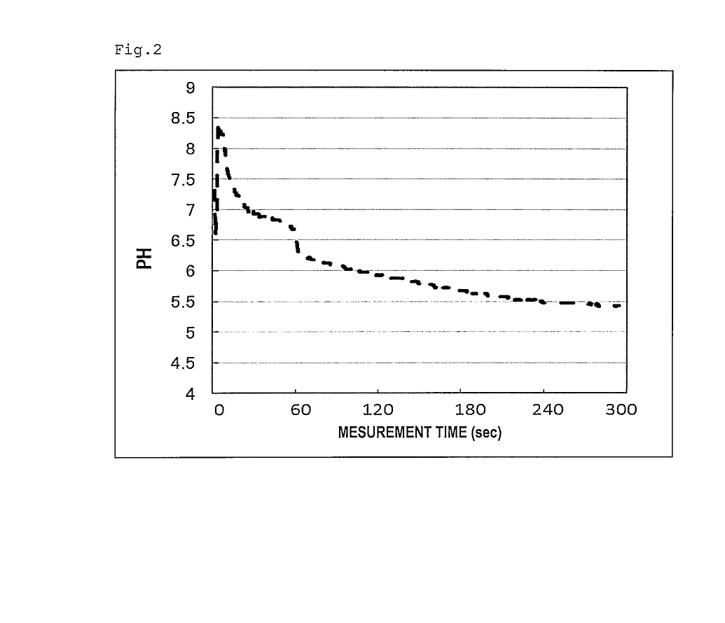

Furthermore, the pH change of the laminated sheet of Example 48 was observed with the measurement method 2. The results are illustrated in FIG. 2.

TABLE 24

| Component | (A) | (B) | (C) |
|---|---|---|---|
| Anhydrous Sodium | 8 | — | — |
| pyrophosphate | | | |
| Phytic acid | — | 15 | — |
| Potassium hydroxide | — | q.s. | — |
| Hydrochloric acid | q.s. | — | — |
| Conc. glycerin | 20 | 10 | — |
| Flavor | 1 | 1 | — |
| Decaglyceryl myristate | 2.5 | 2.5 | — |
| Hydroxypropylmethyl | — | Balance | — |
| cellulose | | | |
| Pullulan | Balance | — | — |
| Polyethylene glycol | — | 10 | — |
| Sucralose | 0.02 | 0.02 | — |
| Blue No. 1 | — | 0.003 | — |
| Ethyl cellulose | — | — | 70 |
| Polyethylene glycol | — | — | 30 |
| Total | 100 | 100 | 100 |
| Amount in terms of acid | 5.4 | 15 | — |
| Thickness (μM) | 8 | 40 | 1 |
| Dissolution time (minute) | 1.5 | 22 | — |
| pH | 9 | 4.5 | 7 |

TABLE 25

| | Water content relative to 100 mass |
|---|---|
| | parts by dry mass of sheet |
| Example 46 | 14 mass parts |
| Example 47 | 6.4 mass parts |
| Example 48 | 5.6 mass parts |
| Example 49 | 3.6 mass parts |

From these results, with respect to the laminated sheets of Examples 46 to 49, pH difference between the layer (A) and the layer (B) was remarkably observed in Example 49 having small water content, but flexibility and adhesion of the sheet were excellent in Example 46 even when the measurement interval was long as illustrated in FIG. 1.

What is claimed is:

1. An adhesive sheet for teeth, which comprises
a layer (A) that has a pH of 6.6 or more and 10.5 or less when the layer (A) is dissolved in water, and
a layer (B) that comprises a component (b) of one or more kinds selected from the group consisting of phytic acid, hexamethaphosphoric acid, an organic acid having a pKa of from 4.5 to 7.0 at 25° C. and salts thereof, and that has a pH of 3.5 or more and 6.5 or less when the layer (B) is dissolved in water.

2. The adhesive sheet for teeth according to claim 1, wherein a time until entire dissolution of the layer (B) in water is longer than a time until entire dissolution of the layer (A) in water.

3. The adhesive sheet for teeth according to claim 1, wherein the layer (A) comprises a component (a) of one or more kinds selected from the group consisting of alkyl sulfuric acid, acylmethyltauric acid, N-acylsarcosic acid, orthophosphoric acid, condensed phosphoric acid and salts thereof.

4. The adhesive sheet for teeth according to claim 1, wherein the layer (A) constitutes the side attached to teeth.

5. The adhesive sheet for teeth according to claim 1, wherein a thickness ratio between the layer (A) and the layer (B) (Aₜ:Bₜ) is from 1:1 to 1:10.

6. The adhesive sheet for teeth according to claim 1, which further comprises a layer (C) that does not collapse during dissolution of the layer (A) and/or the layer (B), and constitutes the opposite side to the side attached to teeth.

7. The adhesive sheet for teeth according to claim 6, wherein the layer (C) comprises, components (c), one or more (co)polymers selected from the group consisting of cellulose acetate phthalate, polyvinyl acetate, ethylcellulose, polymethylmethacrylate, a methacryloylethylbetaine-methacrylate copolymer, a methacrylic acid copolymer and an aminoalkyl methacrylate copolymer.

8. The adhesive sheet for teeth according to claim 1, wherein a total thickness is from 10 μm or more and 1000 μm or less.

9. The adhesive sheet for teeth according to claim 1, wherein a time until entire dissolution of the layer (A) and the layer (B) in water from the time immediately after the attachment to teeth is 1 minute or more and 90 minutes or less.

10. The adhesive sheet for teeth according to claim 1, wherein a time ratio until entire dissolution between layer (A) and the layer (B) (Aₘ:Bₘ) is from 1:1 to 1:15.

11. The adhesive sheet for teeth according to claim 1, which comprises a middle layer (X) between the layer (A) and the layer (B).

12. The adhesive sheet for teeth according to claim 1, wherein the layer (B) constitutes the side attached to teeth.

13. The adhesive sheet for teeth according to claim 1, wherein the layer (A) is formed using a base material solution (A) with a pH of 6.6 or more and 10.5 or less and the layer (B) is formed using a base material solution (B) with a pH of 3.5 or more and 6.5 or less.

14. The adhesive sheet for teeth according to claim 3, wherein a content of the component (a) in the layer (A) is 1% by mass or more and 15% by mass or less by dry weight, and a content of the component (b) in the layer (B) is 5% by mass or more and 30% by mass or less by dry weight.

15. The adhesive sheet for teeth according to claim 1, wherein a water content in the adhesive sheet for teeth is 18 mass parts or less relative to 100 mass parts by dry mass of the adhesive sheet for teeth, and the adhesive sheet for teeth is sealed in a packaging bag.

16. A method for whitening teeth comprising:
applying an adhesive sheet for teeth to teeth to apply a layer (A) and a layer (B) to the teeth by dissolving the layers in saliva,
wherein the adhesive sheet comprises
a layer (A) that has a pH of 6.6 or more and 10.5 or less when the layer (A) is dissolved in water, and
a layer (B) that comprises a component (b) of one or more kinds selected from phytic acid, hexamethaphosphoric acid, an organic acid having a pKa of from 4.5 to 7.0 at 25° C. and salts thereof, and that has a pH of 3.5 or more and 6.5 or less when the layer (B) is dissolved in water.

17. The method for whitening teeth according to claim 16, wherein the layer (A) comprises a component (a) of one or more kinds selected from alkyl sulfuric acid, acylmethyltauric acid, N-acylsarcosic acid orthophosphoric acid, condensed phosphoric acid and salts thereof.

18. The method for whitening teeth according to claim 16, further comprising attaching the side of the layer (A) to teeth.

19. The method for whitening teeth according to claim 17, further comprising attaching the side of the layer (A) to teeth.
